SEQUENCE LISTING (14 sequences)

SEQ ID NO: 1 - DNA, 2297 bp, Murinae gen. sp.
cggaaccccgggagccagggtccccagcatgatcctcggcagcctgagccgggcggggcccctccctctgctccggcagccccccatcatgcagccaccgatggacctcaagcagatcctgcccttcccactagagccagccccaaccctgggcctcttcagcaactacagcacaatggaccctgtacagaaagctgtgctctcccacacttttggaggacccttgctcaagaccaagcggccagtcatttcctgtaatgtctgtcagatccgcttcaattctcagagccaggctgaggcgcactacaagggtaatcgccatgcccgaagagtcaaaggcatcgaagctgccaaaacccgaggcagggagcctagtgtccgggaatcaggagatccagctccagcaggcagcatccctccgagtggggatggtgtagcccctcgtccagtttccatggagaatggcctgggtccagctccaggatccccagagaaacagcctggctccccatcccctcccagtgttccagagtcgggacagggtgtaaccaagggtgaagggggaacttcagtcccagcttccctgcctgggggtagcaaggaagaggaggagaaggctaagcgtctgctctactgtgcactgtgcaaggtggctgtgaactccctgtcccagcttgaggcacataacaaaggtactaagcacaagacaattttggaggccggaagtgggctgggagccatcaaagcttaccctcgggtggggccaattctggggaaccagaggctcctgcccagggacggaaccttccactgtgagatctgcaatgtcaaggtcaattcggaggtccagctgaaacagcacatctccagcaggaggcaccgagatggcgtggctgggaagcccaaccctctactgagccggcacaagaagcctaggggcgctgcagagctggcgggcacgctgactttctcaaaggagctgcccaagtccctggccggtggcctgctccccagccccctagcggtggctgcggtgatggccgctgcagcaggatctccgctgtccctgcgtccagctccagctgcacctcttctgcagggaccaccgatcacacaccctctactccaccctgccccaggacccatccgaactgcgcacggacccatcctcttctccccctactgacctcaaccctgaacccctcccattgaatcccccacctccagccgggacccaggtctccgggctcccagcccgcccctcttcccggcgctccctgaatggtctctccttccccccaccccgaggtacggggttccaggaaaggggaggggtagcgggggaggggggcttcagaagggggggaacaccccagatctcaggaaccccgccccctgcccttccctctcccctagaaaagaggggggccgtctcacccccgagccccctcggagacaccccctcccaaaagccatgtccatccaacccttcccctccaaacctagcacaaaacggggttcacaagccatggtcggggcccggggggagaacatggattttcttggcaataagcggactctgggactccggctcccctaccccaaactaaagcgcttccgtgaacaaccccatcctcccgcaggaggaggggaacaggcgggatcctgggtccctcgtaagcactttggttttaccgcctgcaacctcactgtgcccgccccgcaccatgccccagccccgggtccagtcgggcccatcgcagggggcagcgcttgggggcatctcaggcacttgggtgggaccaaggagatgccctcatagacccttccctcgccttcttcctccccggtccgggttccattcttttcaccagcacccatcgcccaaggggtaccaaggggggcaaggggtgtccagtccaagcccacccccgcctcgccttccgcaaaactgtgagcaaaaagcaatagaagcctcgcccccgcccccggcccgtccctttccgcaggatttgccgtctgtagcctccccattccagttcctagacctcatggctgccccctcccgtgacacctccactgcacaactcgggcgggggcgtgacccccaacccgtcccttctgtggtttccgtgtggtcagcccttccagccgccgacccgggatggggactggcccccactggccctcccctccccatggactctttctgcttgacaatgtagcaaaccccctccctctttctctctctttgacaataaagtctggatttgttctgccctctcgccaaggaaaaa

SEQ ID NO: 2 - protein, 385 aa, Murinae gen. sp.
MILGSLSRAGPLPLLRQPPIMQPPMDLKQILPFPLEPAPTLGLFSNYSTMDPVQKAVLSHTFGGPLLKTKRPVISCNVCQIRFNSQSQAEAHYKGNRHARRVKGIEAAKTRGREPSVRESGDPAPAGSIPPSGDGVAPRPVSMENGLGPAPGSPEKQPGSPSPPSVPESGQGVTKGEGGTSVPASLPGGSKEEEEKAKRLLYCALCKVAVNSLSQLEAHNKGTKHKTILEAGSGLGAIKAYPRVGPILGNQRLLPRDGTFHCEICNVKVNSEVQLKQHISSRRHRDGVAGKPNPLLSRHKKPRGAAELAGTLTFSKELPKSLAGGLLPSPLAVAAVMAAAAGSPLSLRPAPAAPLLQGPPITHPLLHPAPGPIRTAHGPILFSPY

SEQ ID NO: 3 - DNA, 1258 bp, Murinae gen. sp.
ctcgggcgtctgacagagttgttgttccggcacgccgcggagacgtgagttgttccaggtggatgtgggcagagaggtttgcagaactgaaatggaggtcggagcttcgttccagaaggttagtgggtcatctgattctgtggccacactgaacagtgaagaatttgttttggtttctcagcacacagatgccacttctataaaggatgatgggaagccacagctgaagatagcttccaatggtgatgagcagttggaaaaagccatggaagagattttgagagattccgagaaaggacaaagcggtctacctgttgattgccaaggatccagtgagatttcagactgtccttttggagatgtgccggccagccaaacaactaagccgcctctccagttaattttggatccatctaatacagaaatttccacacccagaccatcttctccaagcagatttcctgaagaagacagtgttctctttaacaagctgacatacttaggatgtatgaaggtttcttcccacgcagtgaagtggaggctttacggccatgccaccatgagagcttccagtcagtacccctttgctgttactctgtatgtgcccaatgttccagaaggatctgtgagaatcatagaccagtcaagcaatgtggagatagcatcttttccaatttataaagtgcttttctgtgcacgtgggcatgatgagacagccgagagcaattgctttgcatttacagagagttctcatggctcagaagaatttcagatacatgttttctcctgtgaaattaaagaggcagtaagcagaattttatatagtttctgcactgcattcaaacgttcttccagacaagtgtctgatgttaaagactcagtcatcccgactcccgacagtgatgtgtttaccttcagtgtctccttggaggtcaaagaagatgatggaaaaggaaactttagtcccgtgcctaaggatagagataaattttatatcaaaataaagcaaggaatagagaagaaggttgtgattacagttcagcaactgtctaacaaagaattagctattgagagatgttttggaatgttattaagcccaggtcgaaacgtgaagaacagtgacatgcatttactggatatggagtcccggaaagagctatgatgggagagcttacgtcatcacgggcatgtggaaccccaacgcaccaatatttctggctcttaatgaagaaaccctgtctcaaaaaaaaccaaaaaa

SEQ ID NO: 4 - protein, 298 aa, Murinae gen. sp.
MEEILRDSEKGQSGLPVDCQGSSEISDCPFGDVPASQTTKPPLQLILDPSNTEISTPRPSSPSRFPEEDSVLFNKLTYLGCMKVSSHAVKWRLYGHATMRASSQYPFAVTLYVPNVPEGSVRIIDQSSNVEIASFPIYKVLFCARGHDETAESNCFAFTESSHGSEEFQIHVFSCEIKEAVSRILYSFCTAFKRSSRQVSDVKDSVIPTPDSDVFTFSVSLEVKEDDGKGNFSPVPKDRDKFYIKIKQGIEKKVVITVQQLSNKELAIERCFGMLLSPGRNVKNSDMHLLDMESRKEL

SEQ ID NO: 5 - DNA, 21 bp, Artificial Sequence
  Description of Artificial Sequenceprimer
gcagagctctgggcggcgggt

SEQ ID NO: 6 - DNA, 21 bp, Artificial Sequence
  Description of Artificial Sequenceprimer
ctcgggcgtctgacagagttg

SEQ ID NO: 7 - DNA, 18 bp, Artificial Sequence
  Description of Artificial Sequenceprimer
cccgggacagaacgacgc

SEQ ID NO: 8 - DNA, 18 bp, Artificial Sequence
  Description of Artificial Sequenceprimer
cggaccccgggagccagg

SEQ ID NO: 9 - DNA, 21 bp, Artificial Sequence
  Description of Artificial Sequenceprimer
attacgccagctggcgaaagg

SEQ ID NO: 10 - DNA, 21 bp, Artificial Sequence
  Description of Artificial Sequenceprimer
gattggtggcgacgactcctg

SEQ ID NO: 11 - DNA, 21 bp, Artificial Sequence
  Description of Artificial Sequenceprimer
tatcttcagctgtggcttccc

SEQ ID NO: 12 - DNA, 30 bp, Artificial Sequence
  Description of Artificial Sequenceprimer
ccgtcgactctggcgccgctgctctgtcag

SEQ ID NO: 13 - DNA, 19 bp, Artificial Sequence
  Description of Artificial Sequenceprimer
cggaaccccgggagccagg

SEQ ID NO: 14 - DNA, 22 bp, Artificial Sequence
  Description of Artificial Sequenceseq14
tctggggatcctggagctggac

(19) United States
(12) Patent Application Publication
(10) Pub. No.: US 2002/0102724 A1
(43) Pub. Date: Aug. 1, 2002

(54) Novel hematopoietic genes and polypeptides

(76) Inventors: Michihiro Hidaka, Kumamoto (JP); William Stanford, Toronto (CA); Georgina Caruana, Melbourne (AU); Yuki Kimura, Toronto (CA)

Correspondence Address: MERCHANT & GOULD PC, P.O. BOX 2903, MINNEAPOLIS, MN, 55402-0903, US

(21) Appl. No.: 09/934,332

(22) Filed: Aug. 17, 2001

Related U.S. Application Data
(63) Continuation of application No. PCT/CA00/00171, filed on Feb. 18, 2000.
(60) Provisional application No. 60/120,972, filed on Feb. 19, 1999.

(51) Int. Cl. C12Q 1/68; G01N 33/573; C07H 21/04; C12P 21/06; C12N 15/82; C12N 15/09; C12N 15/29; C07K 1/00; C07K 14/00; C12N 5/04; C07K 16/00

(52) U.S. Cl. 435/320.1; 536/23.6; 536/24.1; 435/419; 530/350; 435/69.1; 800/278; 424/130.1; 536/24.3; 435/6; 435/7.1; 435/7.4; 800/13

(57) ABSTRACT

The invention also relates to novel genes primarily expressed in hematopoietic lineages, polypeptides encoded by the novel genes and truncations, analogs, homologs, and isoforms of the polypeptides; and, uses of the polypeptides and genes.

### FIELD OF THE INVENTION

The invention relates to novel genes primarily expressed in hematopoietic cells, polypeptides encoded by the novel genes and truncations, analogs, homologs, and isoforms of the polypeptides; and, uses of the polypeptides and genes.

### BACKGROUND OF THE INVENTION

Many of the molecular events involved in the embryonic development and differentiation of hematopoietic cells remain to be elucidated. In the mouse, several genetic approaches have been employed in an attempt to identify and determine the function of novel hematopoietic genes. One approach involves the cloning of mutant loci responsible for altered hematopoietic phenotypes in mice harboring naturally occurring or induced mutations [1, 2, 3]. A complementary approach involves the generation of new mutations in the mouse germline by homologous recombination in embryonic stem (ES) cells to mutate candidate hematopoietic genes [4, 5, 6, 7, 8, 9, 10]. Both approaches are labour intensive, the former being restricted to the repetoire of mutant phenotypes while the latter is restricted to previously identified genes. Thus, neither approach is easily applied on a genome-wide level. In contrast, gene trapping in ES cells provides an effective approach to screen and analyze novel genes because it simultaneously provides information on the pattern of expression in vivo, sequence and resulting mouse mutant phenotype [11, 12, 13, 14, 15, 16, 17, 18].

One potential drawback of gene trapping is the very large size of the mammalian genome and hence the integration of the gene trap vector into genes that may not be of immediate interest. To pre-select for genes that are preferentially expressed in hematopoietic cells, an expression trapping strategy has been developed that takes advantage of the developmental potential of ES cells to differentiate into diverse cell lineages in vitro, including hematopoietic cells [19, 20, 21, 22, 23, 24, 25]. Upon the removal of leukemia inhibitory factor (LIF), ES cells spontaneously differentiate into embryoid bodies (EBs), structures which resemble postimplantation embryos, comprising a number of cell lineages and structures, including blood islands which contain hematopoietic precursor cells. Secondary plating of cells obtained from dissociated EBs in semisolid cultures, containing various cytokines and growth factors, results in the growth of colonies of erythroid, myeloid and lymphoid lineages [19, 24, 25, 26, 27, 28]. Recently, in vitro differentiation of ES cells has been combined with gene trapping strategies to pre-screen for novel genes which respond to exogenous factors [29, 17] or are regulated in a tissue-specific manner prior to transmission into the mouse germline [18, 30, 31, 32]. In order to screen large numbers of cell clones based on the expression of trapped genes in hematopoietic and endothelial cells, an expression trap screen has been combined with an attached EB culture system to identify genes expressed within blood islands and endothelial cells [18]. Muth et al.,(1998) [32] have developed a related approach to identify genes that are repressed during hematopoietic differentiation.

### SUMMARY OF THE INVENTION

The ES cell-OP9 co-culture system has been combined with gene trapping to screen for genes involved in the development and differentiation of hematopoietic cells. The OP9 stromal cell line induces ES cell differentiation into mesodermal colonies that, when replated, differentiate into single lineage precursors, without the addition of exogenous growth factors [33]. Using this system, the present inventors have identified two novel genes designated Hzf (hematopoietic zinc finger) and Hhl (denoting the embryonic expression seen within hematopoietic cells, heart and liver) which are expressed in a regulated pattern during hematopoietic differentiation in vitro. These genes exhibit hematopoietic-specific expression in vivo, indicating that the in vitro pre-screening strategy successfully predicts the expression of trapped genes in vivo. Within the hematopoietic compartment, both genes were predominantly expressed in megakaryocytes.

The in vivo function of Hzf was investigated by gene targeting, demonstrating that Hzf is essential for megakaryopoiesis and for hemostasis in vivo. Hzf-deficient mice exhibited a pronounced tendency to rebleed and had reduced α-granule substances in both megakaryocytes and platelets, and the production of large, faintly stained platelets. The results indicate that Hzf plays important roles in regulating the synthesis of α-granule substances and/or their packing into α-granules during the process of megakaryopoiesis.

Broadly stated the present invention contemplates an isolated hematopoietic nucleic acid molecule encoding a Hzf or Hhl polypeptide of the invention, including mRNAs, DNAs, cDNAs, genomic DNAs, PNAs, as well as antisense analogs and biologically, diagnostically, prophylactically, clinically or therapeutically useful variants or fragments thereof, and compositions comprising same.

The invention also contemplates isolated hematopoietic Hzf or Hzl polypeptides encoded by a nucleic acid molecule of the invention a truncation, an analog, an allelic or species variation thereof, or a homolog of a polypeptide of the invention or a truncation thereof. (Truncations, analogs, allelic or species variations, and homologs are collectively referred to herein as “Hzf Related Polypeptides” or “Hhl Related Polypeptides).

The nucleic acid molecules of the invention permit identification of untranslated nucleic acid sequences or regulatory sequences that specifically promote expression of genes operatively linked to the promoter regions. Identification and use of such promoter sequences are particularly desirable in instances, such as gene transfer or gene therapy, which may specifically require heterologous gene expression in a limited environment e.g hematopoietic system. The invention therefore contemplates a nucleic acid molecule comprising a non-coding sequence such as a 5′ and/or 3″ sequence, preferably a non-coding sequence of Hzf or Hhl.

The nucleic acid molecules which encode for the mature Hzf or Hhl polypeptide may include only the coding sequence for the mature polypeptide; the coding sequence for the mature polypeptide and additional coding sequences (e.g. leader or secretory sequences, propolypeptide sequences); the coding sequence for the mature polypeptide (and optionally additional coding sequence) and non-coding sequence, such as introns or non-coding sequence 5′ and/or 3′ of the coding sequence of the mature polypeptide.

Therefore, the term “nucleic acid molecule encoding a polypeptide” encompasses a nucleic acid molecule which includes only coding sequence for the polypeptide as well as a nucleic acid molecule which includes additional coding and/or non-coding sequences.

The nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule.

Vectors are contemplated within the scope of the invention which comprise regulatory sequences of the invention, as well as chimeric gene constructs wherein a regulatory sequence of the invention is operably linked to a heterologous nucleic acid, and a transcription termination signal.

A vector can be used to transform host cells to express a Hzf or Hhl Polypeptide or a Hzf or Hhl Related Polypeptide, or a heterologous polypeptide (i.e. a polypeptide not naturally in the host cell). Therefore, the invention further provides host cells containing a vector of the invention. The invention also contemplates transgenic non-human mammals whose germ cells and somatic cells contain a vector comprising a nucleic acid molecule of the invention in particular one that encodes an analog of Hzf or Hhl, or a truncation of Hzf or Hhl.

The polypeptides of the invention may be obtained as an isolate from natural cell sources, but they are preferably produced by recombinant procedures. In one aspect the invention provides a method for preparing a Hzf or Hhl Polypeptide, or a Hzf or Hhl Related Polypeptide utilizing the purified and isolated nucleic acid molecules of the invention. In an embodiment a method for preparing a Hzf or Hhl Polypeptide, or a Hzf or Hhl Related Polypeptide is provided comprising:

(a) transferring a vector of the invention comprising a nucleic acid sequence encoding a Hzf or Hhl Polypeptide, or a Hzf or Hhl Related Polypeptide, into a host cell;

(b) selecting transformed host cells from untransformed host cells;

(c) culturing a selected transformed host cell under conditions which allow expression of the Hzf or Hhl Polypeptide, or a Hzf or Hhl Related Polypeptide; and

(d) isolating the Hzf or Hhl Polypeptide, or Hzf or Hhl Related Polypeptide.

The invention further broadly contemplates a recombinant Hzf or Hhl Polypeptide, or Hzf or Hhl Related Polypeptide obtained using a method of the invention.

A Hzf or Hhl Polypeptide, or Hzf or Hhl Related Polypeptide of the invention may be conjugated with other molecules, such as polypeptides, to prepare fusion polypeptides or chimeric polypeptides. This may be accomplished, for example, by the synthesis of N-terminal or C-terminal fusion polypeptides.

The invention further contemplates antibodies having specificity against an epitope of a Hzf or Hhl Polypeptide, or a Hzf or Hhl Related Polypeptide of the invention. Antibodies may be labeled with a detectable substance and used to detect polypeptides of the invention in biological samples, tissues, and cells.

The invention also permits the construction of nucleotide probes that are unique to nucleic acid molecules of the invention and/or to polypeptides of the invention. Therefore, the invention also relates to a probe comprising a sequence encoding a polypeptide of the invention, or a portion (i.e. fragment) thereof. The probe may be labeled, for example, with a detectable substance and it may be used to select from a mixture of nucleic acid molecules a nucleic acid molecule of the invention including nucleic acid molecules coding for a polypeptide which displays one or more of the properties of a polypeptide of the invention.

In accordance with an aspect of the invention there is provided a method of, and products for diagnosing and monitoring conditions mediated by Hzf or Hhl by determining the presence of nucleic acid molecules and polypeptides of the invention.

The invention also provides antisense nucleic acid molecules e.g. by production of a mRNA or DNA strand in the reverse orientation to a sense molecule. An antisense nucleic acid molecule may be used to suppress Hzf or Hhl expression in a cell.

Still further the invention provides a method for evaluating a test substance or compound for its ability to modulate the biological activity of a Hzf or Hhl Polypeptide, or a Hzf or Hhl Related Polypeptide of the invention. For example, a substance or compound which inhibits or enhances the activity of a Hzf or Hhl Polypeptide, or a Hzf or Hhl Related Polypeptide may be evaluated. “Modulate” refers to a change or an alteration in the biological activity of a polypeptide of the invention. Modulation may be an increase or a decrease in activity, a change in characteristics, or any other change in the biological, functional, or immunological properties of the polypeptide.

Compounds which modulate the biological activity of a polypeptide of the invention may also be identified using the methods of the invention by comparing the pattern and level of expression of a nucleic acid molecule or polypeptide of the invention in biological samples, tissues and cells, in the presence, and in the absence of the compounds.

Methods are also contemplated that identify compounds or substances (e.g. polypeptides) which interact with Hzf or Hhl regulatory sequences (e.g. promoter sequences, enhancer sequences, negative modulator sequences).

The nucleic acid molecules, polypeptides, and substances and compounds identified using the methods of the invention, may be used to modulate the biological activity of a Hzf or Hhl Polypeptide, or a Hzf or Hhl Related Polypeptide of the invention, and they may be used in the treatment of conditions mediated by Hzf or Hhl such as hematopoietic disorders. Accordingly, the nucleic acid molecules, polypeptides, substances and compounds may be formulated into compositions for administration to individuals suffering from one or more of these conditions. Therefore, the present invention also relates to a composition comprising one or more of a polypeptide, nucleic acid molecule, or substance or compound identified using the methods of the invention, and a pharmaceutically acceptable carrier, excipient or diluent. A method for treating or preventing these conditions is also provided comprising administering to a patient in need thereof, a composition of the invention.

The present invention in another aspect provides means necessary for production of gene-based therapies directed at the hematopoietic system. These therapeutic agents may take the form of polynucleotides comprising all or a portion of a nucleic acid molecule of the invention comprising a regulatory sequence of Hzf or Hhl placed in appropriate vectors or delivered to target cells in more direct ways.

In accordance with a further aspect of the invention, there are provided processes for utilizing polypeptides or nucleic acid molecules, for in vitro purposes related to scientific research, synthesis of DNA and manufacture of vectors.

In other embodiments, the invention provides a method for identifying inhibitors or enhancers of a Hzf or Hhl Polypeptide interaction, comprising

(a) providing a reaction mixture including the Hzf or Hhl Polypeptide and a substance that associates with the Hzf or Hhl Polypeptide, or at least a portion of each which interact;

(b) contacting the reaction mixture with one or more test compounds;

(c) identifying compounds which inhibit or potentiate the interaction of the Hzf or Hhl Polypeptide and substance.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as those isolated from animals, plants, fungus and/or microbes.

In an embodiment the invention provides a method for identifying a compound which inhibits or enhances the interaction of a Hzf or Hhl Polypeptide and a substance that forms a complex with the Hzf or Hhl Polypeptide comprising the steps of:

(a) contacting a Hzf or Hhl Polypeptide with the substance and a test compound under conditions which permit the formation of complexes between the Hzf or Hhl Polypeptide and the substance;

(b) assaying for complexes;

(c) performing a control experiment in which said parts (a) and (b) are performed in the absence of said test compound; and

(d) comparing the effect of said test compound to the effect of the control experiment to determine if said test compound inhibits or enhances the interaction between the Hzf or Hhl Polypeptide and substance.

Still another aspect of the present invention provides a method of conducting a drug discovery business comprising:

(a) providing one or more assay systems for identifying agents by their ability to inhibit or potentiate the interaction of a Hzf or Hhl Polypeptide and a substance that binds to the polypeptide;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

These and other aspects, features, and advantages of the present invention should be apparent to those skilled in the art from the following drawings and detailed description.

### DESCRIPTION OF THE DRAWINGS

The invention will be better understood with reference to the drawings in which:

FIG. 1. LacZ Expression Pattern of Clone Hhl (A-D) and Hzf (E-F) during in vitro Hematopoietic Differentiation on OP9 stromal cells. β-galactosidase (β-gal) activity within the gene trap clone Hhl was detected around day 5 within mesodermal colonies and was maintained in hematopoietic cells (day 12). The gene trap clone Hzf expressed low levels of β-gal activity in undifferentiated ES cells which was maintained within hematopoietic cells (day 2-12). Cultures were fixed with 0.25% gluteraldehyde and stained with X-gal after 2 days (A, E), 5 days (B, F), 8 days (C, G) and 12 days (D, H). Bar=50 mm.

FIG. 2. LacZ Expression in Hhl (A-D) and Hzf (E-H) Heterozygous Embryos. Wholemount lacZ expression within 9.5 d.p.c (A) Hhl heterozygous F1 embryos demonstrated expression within the yolk sac and heart and also within the liver at 14.5 d.p.c (B). Histological analysis of the liver (C) and heart (D) of 14.5 d.p.c Hhl embryos. Wholemount lacZ expression within 9.5 d.p.c Hhl F1 heterozygous embryos (E) demonstrated expression within the somites, basal ganglia, apical ectodermal region of the limb buds and liver primordium and in the skin (FIG. 2H). At 14.5 d.p.c (F) expression was seen in the trigeminal ganglia, thymus, salivary gland, spinal cord and spotty staining within the fetal liver. Histological analysis of the liver (G) and skin (H) of 14.5d.p.c Hzf embryo. Bar=100 mm (A,C,E,H), 1 mm (B, F), 500 mm (D), 50 mm (G).

FIG. 3. LacZ Expression within Bone Marrow Cells derived from Hhl and Hzf Heterozygous Adult Mice. X-gal staining of Hhl (A) and Hzf (B) bone marrow cells. LacZ expression detected by FDG staining (C) and cell morphology analysed by Wright-Giemsa staining (D) of the same sections of bone marrow cells from Hzf mice demonstrating the coincidence of lacZ expression within cells morphologically resembling megakaryocytes. Hhl bone marrow sections also demonstrated the same coincidence of expression within megakaryocytes (not shown). Bar=50 mm.

FIG. 4. LacZ Expression within Bone Marrow Cell derived Colonies. Bone marrow cells from Hzf and Hhl adult heterozygous mice were cultured in semi-solid agarose in the presence of TPO, IL-3 and SLF for the analysis of CFU-Mk. For the analysis of BFU-E, CFU-G, M, GM and GEMM bone marrow cells were plated in methylcellulose containing IL-3, IL-6, Epo and SLF. Colonies were either X-gal stained directly in the cultures or picked and then stained. High levels of X-gal staining were seen in CFU-Mk and CFU-GEMM for both Hzf and Hhl derived colonies. X-gal staining was seen in Hhl derived BFU-E but not in those derived from Hzf bone marrow cells. A low frequency of X-gal staining was detected in the macrophage component (depicted by the arrows) of CFU-M and GM for Hhl and only in CFU-M for Hzf. Photographs of cells from representative colonies are shown.

FIG. 5. 5′-RACE Sequence of Hhl and Hzf.(5A) Schematic diagram of the PT1/ATG gene trap vector and 5′-RACE methodology. (5B) The PT1/ATG vector contains a promoterless lacZ gene immediately upstream of a splice acceptor (SA) site and the neoR gene driven by the PGK-1 promoter. Random integration within an intron will generate a spliced fusion transcript between lacZ and the endogenous gene located at the site of vector integration. The fusion with lacZ enables primers to be designed (a, b and e ) to clone part of the upstream fused exon of the trapped gene by 5′ rapid amplification of cDNA ends (RACE). The Kpn1 and Pst 1 restriction sites and primers (c & d, GTlacz-3 and 4, respectively) used in the inverse PCR strategy for Hhl are also indicated. (5C) 5′- RACE sequence of Hhl (bold). Arrows indicate sequences of the 12G10-5 and 12G10-6 primers used for 3′-RACE. (C) 5′-RACE sequence of Hzf (bold). Arrows indicate sequences of 4H5-1 and 4H5-2 primers used for 3′-RACE

FIG. 6. Nucleotide Sequence and the Deduced Amino Acid Sequence of the Hhl and Hzf genes. (6A) The putative initiation codon of Hhl is located at bases 266-268. The Hhl open reading frame (ORF) encodes 298 amino acids. (6B) The longest ORF of Hzf begins with the ATG at nucleotide 29 and encodes 396 amino acids. The polyadenylation signal is underlined and the three C2H2-type zinc finger motifs are boxed. The arrows depict the site of vector integration.

FIG. 7. Northern blot analysis of total RNA from wild type mouse adult tissues using probes from the Hhl (A) and Hzf (B) genes. The GAPDH probe was used as an internal loading control (C).

FIG. 8. RT-PCR Analysis of Hzf and Hhl Expression in Normal and Trapped Mice. RNA was isolated from brain and amplified by RT-PCR from genotyped Hzf and Hhl mice. Amplified products were then southern blotted and hybridized with gene specific or lacZ probes. Amplified products were obtained from Hhl (A) and Hzf (B) homozygous mice using a primer 5′ and another 3′ to the vector integration site demonstrating that endogenous transcript 3′ to the integration site had not been disrupted. Wild-type mice were distinguished from mutant mice due to the lack of amplification of a Hzf or Hhl-lacZ fusion product using a primer 5′ to the integration site and another complementary to the lacZ reporter gene.

FIG. 9. Targeted Disruption of the Hzf Locus (A) A portion of the mouse 129/Sv Hzf wild type locus (top) showing exons (open boxes) and a 6.5 Kb PstI fragment in the wild type allele. The targeting vector (middle) was designed to replace exons encoding three zinc finger domains with an IRES LacZ (closed box) and a neo (hatched box). The mutated Hzf locus (bottom) contains a 4.5 Kb PstI fragment. The positions of the 5′ flanking and 3′ flanking probes used for Southern blot analysis are shown. Positions of PCR primers for the wild type allele (a and b) and the mutant allele (c) are also shown. P, Ks, RI, Kp, C, X, and B represent PstI, KspI, EcoRI, KpnI, ClaI, XhoI, and BamHI sites, respectively. (B) Genomic DNA was isolated from Hzf^{+/+}, Hzf^{+/-}, and Hzf^{-/-} mice, digested with PstI and analyzed by Southern blotting. Wild type (6.5 Kb) and mutant (4.5 Kb) bands are indicated. (C) Northern blot analysis of Hzf expression. Total RNA extracted from the brains (40 μg/lane) was analyzed for the expression of transcripts corresponding to the Hzf gene.

FIG. 10. Growth Retardation and Hemorrhage in Hzf^{-/-} Mice (A) A Hzf^{-/-} mouse compared with a control littermate 4 weeks after birth, demonstrating slightly smaller size. (B) Transverse section of the brain of a 3-week-old Hzf^{-/-} mouse, demonstrating extensive hemorrhage. Magnification, 100×.

FIG. 11. Unstable Plug Formation and Abnormal Platelet Morphology in Hzf^{-/-} Mice (A) Time taken for initial cessation of bleeding. Each symbol represents a single animal (Hzf^{+/+} mice, open squares, n=22; Hzf^{+/-} mice, closed triangles, n=41; Hzf^{-/-} mice, closed squares, n=19). Bars indicate the average bleeding time for each genotype (Hzf^{+/+} mice, 177.0 sec.; Hzf^{+/-} mice, 162.8 sec.; Hzf^{-/-} mice, 290.5 sec.). Box and whisker graphs indicate the median, first and third quartiles, and the total range of bleeding times for each genotype. (B) Rebleeding occurrences in Hzf^{+/+} (white column), Hzf^{+/-} (gray column), and Hzf^{-/-} (black column) mice. Rebleeding occurred in 68.4% of Hzf^{+/-} mice (n=19) as compared with 36.4% (n=22) of Hzf^{+/+} and 36.6% (n=41) of Hzf^{+/-} mice. (C) Peripheral blood smear from Hzf^{+/+} mice demonstrates normal platelets (arrowheads). Magnification, 100×. (D) Peripheral blood smear from Hzf^{-/-} mice reveals large, faintly stained platelets. Representative platelets are highlighted by an arrowhead. Magnification, 100×. (E) Ultrastructural morphology of platelets of peripheral blood from Hzf^{+/+} mice demonstrates normal α-granules. Scale bar=500 nm. (F) Ultrastractural analysis of abnormal platelet morphology in Hzf^{-/-} mice. Platelets, from Hzf^{-/-} mouse a, b, c, and d, reveal numerous vacuoles with reduced α-granules in panels (a), (b), (c), and (d), respectively. In panel (a), scale bar=500 nm. In panels (b), (c), and (d), scale bars=1000 nm. (G) Reduced platelet-vWF in Hzf^{-/-} mice. Whole washed-platelets from control littermates and Hzf mutant mice were used to assay protein levels of vWF. Hzf^{-/-} platelets have reduced protein levels of vWF. The GPIIb, which is a glycoprotein bounded to plasma membranes of platelets, was used to verify equivalent sample loading. This data was representative with comparable results.

FIG. 12. The Presence of Megakaryocytes and Normal Aspects of Megakaryocyte Maturation in Hzf^{-/-} Mice (A and B) Transverse sections through the spleens from Hzf^{+/+} (A) and Hzf^{-/-} (B) mice, demonstrating the presence of megakaryocytes in each. In both (A) and (B), magnification, 100×. (C and D) Microscopic examination of bone marrow smears from Hzf^{+/+} (C) and Hzf^{-/-} (D) mice, showing the presence of megakaryocytes in each. In both (C) and (D), magnification, 100×. (E) Flow cytometric analysis confirms no significant difference of megakaryocyte frequency between control and Hzf^{-/-} mice. The proportion of megakaryocytes to bone marrow cells was performed after staining with 4A5 mAb, to murine megakaryocytes. Six mice, which were age-and sex-matched, were analyzed in each genotype with FACScan. Results are presented as the means±SD. (F) Representative megakaryocyte DNA ploidy in Hzf^{-/-} mice. The proportion of cells in each ploidy class was determined by PI-staining after gated on 4A5-positive cells with FACScan, demonstrating no effect of Hzf-deficiency on endomitosis. Six mice, which were age-and sex-matched, were analyzed in each genotype. This data was representative with comparable results.

FIG. 13. Ultrastructural Analysis of Megakaryocytic Abnormality (A and B) Mature megakaryocytes from the bone marrow of both Hzf^{+/+} (A) and Hzf^{-/-} (B) mice exhibit hyperlobulated nuclei. In both (a) and (b), scale bars=5000 nm. (C) Detail of the cytoplasm of the megakaryocyte shown in (A). Platelet fields or territories are clearly demarcated. Many α-granules are observed. N, nuclei. Scale bar=2500 nm. (D) Detail of the cytoplasm of the megakaryocyte shown in (B). The Hzf^{-/-} megakaryocyte reveals reduced numbers of α-granules, with many vacuoles. N, nucleus. Scale bar=2500 nm. (E) Ultrastructural analysis of α-granules of the megakaryocyte from Hzf^{+/+} mice. Normal dense α-granules, which exhibits distinct zones: dense nucleoid regions and diffuse granular matrixes are observed. Scale bar=500 nm. (F) Ultrastructural analysis of α-granules of the megakaryocytes from Hzf^{-/-} mice. Some vacuoles (arrowheads) and low dense α-granules (arrows) are present in cytoplasm. M, mitochondria, in which cristae are observed. Scale bar=500 nm.

FIG. 14. Reduced Concentrations of α-Granule Substances in Megakaryocytes from Hzf^{-/-} Mice (A) Western immunoblotting analysis reveals reduced levels of vWF, fibrinogen, PDGF-A, and PDGF-B from bone marrow cells in Hzf^{-/-} mice. The acetylcholinesterase (AChE), a house keeping protein expressed specifically in megakaryoctes, was used to verify equivalent sample loading. This data was representative with comparable results. (B and C) Immunogold localization of PDGF-A on thin sections of megakaryocytes in the spleens of Hzf^{+/+} (B) and Hzf^{-/-} (C) mice. (B) Concentrated gold particles are located in the α-granules (arrowheads) and vesicles (arrows) of cytoplasm in megakaryocytes from Hzf^{+/+} mice. Scale bar=500 nm. (C) A few gold particles are present on the vesicles (arrows) of cytoplasm in megakaryocytes from Hzf^{-/-} mice. Partially empty α-granules and vacant α-granules are also observed. Scale bar=500 nm.

FIG. 15. Down-Regulation of Megakaryocyte-Specific Gene Expression in Hzf^{-/-} Mice RT-PCR analysis of megakaryocyte-specific mRNAs from bone marrow Hzf^{+/+} and Hzf^{-/-} mice. Expression of indicated gene expression was determined by RT-PCR with indicated number of cycles and polyacrylamide gel electrophoresis. HPRT signal was used to equivalent cDNA amounts between male wild type and male Hzf mutant.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Nucleic Acid Molecules and Polypeptides of the Invention

Nucleic Acid Molecules

As hereinbefore mentioned, the invention provides isolated Hzf and Hhl nucleic acid molecules. The term “isolated” refers to a nucleic acid (or polypeptide) removed from its natural environment, purified or separated, or substantially free of cellular material or culture medium when produced by recombinant DNA techniques, or chemical reactants, or other chemicals when chemically synthesized. Preferably, an isolated nucleic acid is at least 60% free, more preferably at least 75% free, and most preferably at least 90% free from other components with which they are naturally associated. The term “nucleic acid” is intended to include modified or unmodified DNA, RNA, including mRNAs, DNAs, cDNAs, and genomic DNAs, or a mixed polymer, and can be either single-stranded, double-stranded or triple-stranded. For example, a nucleic acid sequence may be a single-stranded or double-stranded DNA, DNA that is a mixture of single-and double-stranded regions, or single-, double- and triple-stranded regions, single- and double-stranded RNA, RNA that may be single-stranded, or more typically, double-stranded, or triple-stranded, or a mixture of regions comprising RNA or DNA, or both RNA and DNA. The strands in such regions may be from the same molecule or from different molecules. The DNAs or RNAs may contain one or more modified bases. For example, the DNAs or RNAs may have backbones modified for stability or for other reasons. A nucleic acid sequence includes an oligonucleotide, nucleotide, or polynucleotide. The term “nucleic acid molecule” and in particular DNA or RNA, refers only to the primary and secondary structure and it does not limit it to any particular tertiary forms.

In an embodiment of the invention an isolated nucleic acid molecule is contemplated which comprises:

(i) a nucleic acid sequence encoding a polypeptide having substantial sequence identity with the amino acid sequence of SEQ. ID. NO. 2, or 4;

(ii) a nucleic acid sequence complementary to (i);

(iii) a nucleic acid sequence differing from any of (i) or (ii) in codon sequences due to the degeneracy of the genetic code;

(iv) a nucleic acid sequence comprising at least 10, 15, 18, and preferably at least 20 nucleotides capable of hybridizing to a nucleic acid sequence of SEQ. ID. NO. 1 or 3 or to a degenerate form thereof;

(v) a nucleic acid sequence encoding a truncation, an analog, an allelic or species variation of a polypeptide comprising the amino acid sequence of SEQ. ID. NO. 2 or 4; or

(vi) a fragment, or allelic or species variation of (i), (ii) or (iii)

In a specific embodiment, the isolated nucleic acid molecule comprises:

(i) a nucleic acid sequence having substantial sequence identity or sequence similarity with a nucleic acid sequence of SEQ. ID. NO. 1 or 3;

(ii) nucleic acid sequences complementary to (i), preferably complementary to the full nucleic acid sequence of SEQ. ID. NO. 1 or 3;

(iii) nucleic acid sequences differing from any of the nucleic acid sequences of (i) or (ii) in codon sequences due to the degeneracy of the genetic code; or

(iv) a fragment, or allelic or species variation of (i), (ii) or (iii).

Preferably, a purified and isolated nucleic acid molecule of the invention comprises:

(i) a nucleic acid sequence comprising the sequence of SEQ.ID.NO. 1 or 3; wherein T can also be U;

(ii) nucleic acid sequences complementary to (i), preferably complementary to the full nucleic acid sequence of SEQ.ID.NO. 1 or 3;

(iii) a nucleic acid capable of hybridizing under stringent conditions to a nucleic acid of (i) or (ii) and preferably having at least 18 nucleotides; or

(iv) a nucleic acid molecule differing from any of the nucleic acids of (i) to (iii) in codon sequences due to the degeneracy of the genetic code

The term “complementary” refers to the natural binding of nucleic acid molecules under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules.

In a preferred embodiment the isolated nucleic acid comprises a nucleic acid sequence encoded by the amino acid sequence of SEQ. ID. NO. 2 or 4, or comprises the nucleic acid sequence of SEQ. ID. NO. 1 or 3 wherein T can also be U.

The terms “sequence similarity” or “sequence identity” refer to the relationship between two or more amino acid or nucleic acid sequences, determined by comparing the sequences, which relationship is generally known as “homology”. Identity in the art also means the degree of sequence relatedness between amino acid or nucleic acid sequences, as the case may be, as determined by the match between strings of such sequences. Both identity and similarity can be readily calculated (Computational Molecular Biology, Lesk, A. M., ed., Oxford University Press New York, 1988; Biocomputing: Informatics and Genome Projects, Smith, D. W. ed., Academic Press, New York, 1993; Computer Analysis of Sequence Data, Part I, Griffin, A. M., and Griffin, H. G. eds. Humana Press, New Jersey, 1994; Sequence Analysis in Molecular Biology, von Heinje, G., Academic Press, New York, 1987; and Sequence Analysis Primer, Gribskov, M. and Devereux, J., eds. M. Stockton Press, New York, 1991). While there are a number of existing methods to measure identity and similarity between two amino acid sequences or two nucleic acid sequences, both terms are well known to the skilled artisan (Sequence Analysis in Molecular Biology, von Heinje, G., Academic Press, New York, 1987; Sequence Analysis Primer, Gribskov, M. and Devereux, J., eds. M. Stockton Press, New York, 1991; and Carillo, H., and Lipman, D. SIAM J. Applied Math., 48:1073, 1988). Preferred methods for determining identity are designed to give the largest match between the sequences tested. Methods to determine identity are codified in computer programs. Preferred computer program methods for determining identity and similarity between two sequences include but are not limited to the GCG program package (Devereux, J. et al, Nucleic Acids Research 12(1): 387, 1984), BLASTP, BLASTN, and FASTA (Atschul, S. F. et al., J. Molec. Biol. 215:403, 1990). Identity or similarity may also be determined using the alignment algorithm of Dayhoff et al [Methods in Enzymology 91: 524-545 (1983)].

Preferably, the nucleic acids of the present invention have substantial sequence identity using the preferred computer programs cited herein, for example greater than 50%, 60%, 70%, 80%, or 90% sequence identity, and preferably at least 90% , 95%, 96%, 97%, 98%, or 99% sequence identity to the sequence shown in SEQ. ID. NO. 1 or 3.

Isolated nucleic acids encoding a Hzf or Hhl Polypeptide and comprising a sequence that differs from the nucleic acid sequence shown in SEQ. ID. NO. 1 or 3 due to degeneracy in the genetic code are also within the scope of the invention. Such nucleic acids encode equivalent polypeptides but differ in sequence from the sequence of SEQ. ID. NO. 1 or 3 due to degeneracy in the genetic code. As one example, DNA sequence polymorphisms within hzf or hhl may result in silent mutations that do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of Hzf or Hhl Polypeptide. These amino acid polymorphisms are also within the scope of the present invention. In addition, species variations i.e. variations in nucleotide sequence naturally occurring among different species, are within the scope of the invention.

Another aspect of the invention provides a nucleic acid molecule which hybridizes under selective conditions, (e.g. high stringency conditions), to a nucleic acid which comprises a sequence which encodes a Hzf or Hhl Polypeptide of the invention. Preferably the sequence encodes the amino acid sequence shown in SEQ. ID. NO. 2 or 4 and comprises at least 10, preferably at least 15, more preferably at least 18, and most preferably at least 20 nucleotides. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N.Y. (1989), 6.3.1-6.3.6. For example, hybridization may occur at 30° C. in 750 mM NaCl, 75 mM trisodium citrate, and 1% SDS, preferably 37° C. in 500 mM NaCl, 500 mM trisodium citrate, 1% SDS, 35% formamide, and 100 μg/ml denatured salmon sperm DNA (ssDNA), and more preferably 42° C. in 250 mM NaCl, 25 mM trisodium citrate, 1% SDS, 50% formamide, and 200 μg/ml ssDNA. Useful variations on these conditions will be readily apparent to those skilled in the art.

The stringency may be selected based on the conditions used in the wash step. Wash step stringency conditions may be defined by salt concentration and by temperature. Generally, wash stringency can be increased by decreasing salt concentration or by increasing temperature. By way of example, a stringent salt concentration for the wash step is preferably less than about 30 mM NaCl and 3 mM trisodium citrate, and more preferably less than about 15 mM NaCl and 1.5 mM trisodium citrate. Stringent temperature conditions will generally include temperatures of a least about 25° C., more preferably at least about 68° C. In a preferred embodiment, the wash steps will be carried out at 42° C. in 15 mM NaCl, 1.5 mM trisodium citrate, and 0.1% SDS. In a more preferred embodiment the wash steps are carried out at 68° C. in 15 mM NaCl, 1.5 mM trisodium citrate, and 0.1% SDS. Variations on these conditions will be readily apparent to those skilled in the art.

It will be appreciated that the invention includes nucleic acid molecules encoding a Hzf or Hhl Polypeptide, or a Hzf or Hhl Related Polypeptide, including truncations of the polypeptides, allelic and species variants, and analogs of the polypeptides as described herein. In particular, fragments of a nucleic acid of the invention are contemplated that are a stretch of at least about 10, 15, or 18, and most preferably at least 20 nucleotides, more typically at least 50 to 200 nucleotides but less than 2 kb. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labeled nucleic acid probe based on all or part of the nucleic acid sequence shown in SEQ. ID. NO. 1 or 3. The labeled nucleic acid probe is used to screen an appropriate DNA library (e.g. a cDNA or genomic DNA library). For example, a cDNA library can be used to isolate a cDNA encoding a Hzf or Hhl Polypeptide, or a Hzf or Hhl Related Polypeptide by screening the library with the labeled probe using standard techniques. Alternatively, a genomic DNA library can be similarly screened to isolate a genomic clone encompassing a hzf or hhl gene. Nucleic acids isolated by screening of a cDNA or genomic DNA library can be sequenced by standard techniques.

An isolated nucleic acid molecule of the invention that is DNA can also be isolated by selectively amplifying a nucleic acid of the invention. “Amplifying” or “amplification ” refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.). In particular, it is possible to design synthetic oligonucleotide primers from the nucleotide sequence shown in SEQ. ID. NO. 1 or (e.g. SEQ. ID. Nos. 5-14) for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a Hzf or Hhl Polypeptide, or a Hzf or Hhl Related Polypeptide into an appropriate vector which allows for transcription of the cDNA to produce an RNA molecule which encodes a Hzf or Hhl Polypeptide, or a Hzf or Hhl Related Polypeptide. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

A nucleic acid molecule of the invention may be engineered using methods known in the art to alter the Hzf or Hhl encoding sequence for a variety of purposes including modification of the cloning, processing, and/or expression of the gene product. Procedures such as DNA shuffling by random fragmentation and PCR reassembly of gene fragments and synthetic oligonucleotides may be used to engineer the nucleic acid molecules. Mutations may be introduced by oligonucleotide-mediated site-directed mutagenesis to create for example new restriction sites, alter glycosylation patterns, change codon preference, or produce splice variants.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule is a hzf or hhl gene or encodes a Hzf or Hhl Polypeptide, or a Hzf or Hhl Related Polypeptide can be accomplished by expressing the cDNA in an appropriate host cell by standard techniques, and testing the expressed polypeptide in the methods described herein.

A Hzf or Hhl cDNA or cDNA encoding a Hzf or Hhl Polypeptide, or a Hzf or Hhl Related Polypeptide can be sequenced by standard techniques, such as dideoxynucleotide chain termination or Maxam-Gilbert chemical sequencing, to determine the nucleic acid sequence and the predicted amino acid sequence of the encoded polypeptide.

The nucleic acid molecules of the invention may be extended using a partial nucleotide sequence and various PCR-based methods known in the art to detect uptstream sequences such as promoters and regulatory elements. For example, restriction-site PCR which uses universal and nested primers to amplify unknown sequences from genomic DNA within a cloning vector may be employed (See Sarkar, G, PCR Methods Applic. 2:318-322, 1993). Inverse PCR which uses primers that extend in divergent directions to amplify unknown sequences from a circularized template may also be used. The template in inverse PCR is derived from restriction fragments adjacent to known sequences in human and yeast artificial chromosome DNA (See e.g. Lagerstrom, M., at al, PCR Methods Applic. 1:111-119, 1991). Other methods for retrieving unknown sequences are known in the art (e.g. Parker, J. D. et al, Nucleic Acids Res. 19:305-306, 1991). In addition, PCR, nested primers, and PROMOTERFINDER libraries (Clontech, Palo Alto, Calif.) may be used to walk genomic DNA. The method is useful in finding intron/exon junctions and avoids the need to screen libraries.

It is preferable when screening for full-length cDNAs to use libraries that have been size-selected to include larger cDNAs. For situations in which an oligo d(T) library does not yield a full-length cDNA, it is preferable to use random-primed libraries which often include sequences containing the 5′ regions of genes. Genomic libraries may be useful for extending the sequence into 5′ non-translated regulatory regions.

Commercially available capillary electrophoresis systems may be employed to analyse the size or confirm the sequence of PCR or sequencing products. The system may use flowable polymers for electrophoretic separation, four different nucleotide-specific, laser-stimulated fluorescent dyes, and a charge coupled device camera for detection of the emitted wavelengths. Commercially available software (e.g. GENOTYPER and SEQUENCE NAVIGATOR, Perkin-Elmer) may convert the output/light intensity to electrical signal, and the entire process from loading of samples, and computer analysis and electronic data display may be computer controlled. This procedure may be particularly useful for sequencing small DNA fragments which may be present in limited amounts in a particular sample.

In accordance with one aspect of the invention, a nucleic acid is provided comprising a hzf or hhl regulatory sequence such as a promoter sequence.

The invention contemplates nucleic acid molecules comprising all or a portion of a nucleic acid molecule of the invention comprising a regulatory sequence of a Hzf or Hhl contained in appropriate vectors. The vectors may contain heterologous nucleic acid sequences. “Heterologous nucleic acid” refers to a nucleic acid not naturally located in the cell, or in a chromosomal site of the cell. Preferably, the heterologous nucleic acid includes a nucleic acid foreign to the cell.

In accordance with another aspect of the invention, the nucleic acid molecules isolated using the methods described herein are mutant hzf or hhl gene alleles. For example, the mutant alleles may be isolated from individuals either known or proposed to have a genotype which contributes to the symptoms of a hematopoietic disorder. Mutant alleles and mutant allele products may be used in therapeutic and diagnostic methods described herein. For example, a cDNA of a mutant Hzf or Hhl gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal Hzf or Hhl gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant hzf or hhl allele. Gene products from putatively mutant tissue may be expressed and screened, for example using antibodies specific for a Hzf or Hhl Polypeptide, or a Hzf or Hhl Related Polypeptide as described herein. Library clones identified using the antibodies can be purified and subjected to sequence analysis.

Antisense molecules and ribozymes are contemplated within the scope of the invention. They may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding Hzf or Hhl Polypeptide. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Polypeptides of the Invention

The polypeptides of the invention are primarily expressed in hematopoietic lineages. Hzf contains three C₂H₂-type zinc fingers and it is primarily expressed in megakaryocytes and multipotential progenitors. Hhl is expressed in most myeloid lineages with particularly high expression in multipotential progenitors, erythroid, and megakaryocytic cells.

Amino acid sequences of polypeptides of the invention comprise the sequences of SEQ. ID. NO.2., or SEQ. ID. NO. 4. In addition to the amino acid sequences as shown SEQ. ID. NO.2, or SEQ. ID. NO. 4, the polypeptides of the present invention include truncations of the polypeptides of the invention, and analogs, and homologs of the polypeptides and truncations thereof as described herein.

Truncated polypeptides may comprise peptides having an amino acid sequence of at least five consecutive amino acids in SEQ.ID. NO. 2 or 4 where no amino acid sequence of five or more, six or more, seven or more, or eight or more, consecutive amino acids present in the fragment is present in a polypeptide other than a Hzf or Hhl Polypeptide. In an embodiment of the invention the fragment is a stretch of amino acid residues of at least 10 to 100, preferably at least 10 to 75, more preferably 10 to 50, and most preferably 12 to 20 contiguous amino acids from particular sequences such as the sequences shown in SEQ.ID. NO. 2 or 4. The fragments may be immunogenic and preferably are not immunoreactive with antibodies that are immunoreactive to polypeptides other than Hzf or Hhl.

The truncated polypeptides may have an amino group (-NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated polypeptides may have a carboxyl group, an amido group, a T-butyloxycarbonyl group, or a macromolecule including but not limited to lipid-fatty acid conjugates, polyethylene glycol, or carbohydrates at the carboxy terminal end.

The polypeptides of the invention may also include analogs, and/or truncations thereof as described herein, which may include, but are not limited to the polypeptides, containing one or more amino acid substitutions, insertions, and/or deletions. Amino acid substitutions may be of a conserved or non-conserved nature. Conserved amino acid substitutions involve replacing one or more amino acids with amino acids of similar charge, size, and/or hydrophobicity characteristics. When only conserved substitutions are made the resulting analog should be functionally equivalent to the native polypeptide. Non-conserved substitutions involve replacing one or more amino acids with one or more amino acids which possess dissimilar charge, size, and/or hydrophobicity characteristics.

One or more amino acid insertions may be introduced into a polypeptide of the invention. Amino acid insertions may consist of single amino acid residues or sequential amino acids ranging from about 2 to 15 amino acids in length.

Deletions may consist of the removal of one or more amino acids, or discrete portions from the polypeptide sequence. The deleted amino acids may or may not be contiguous. The lower limit length of the resulting analog with a deletion mutation is about 10 amino acids, preferably 100 amino acids.

An allelic variant at the polypeptide level differs from another polypeptide by only one, or at most, a few amino acid substitutions. A species variation of a polypeptide of the invention is a variation which is naturally occurring among different species of an organism.

The polypeptides of the invention also include homologs and/or truncations thereof as described herein. Such homologs include polypeptides whose amino acid sequences are comprised of the amino acid sequences of regions from other species that hybridize under selective hybridization conditions (see discussion of selective and in particular stringent hybridization conditions herein) with a probe used to obtain a polypeptide of the invention. These homologs will generally have the same regions which are characteristic of a polypeptide of the invention. It is anticipated that a polypeptide comprising an amino acid sequence which is at least 75% identity or at least 80% similarity, preferably 80 to 90% identity or 90% similarity, more preferably 90 to 95% identity or 95% similarity, and most preferably 95 to 99% identity or 99% similarity with an amino acid sequence of SEQ. ID. NO. 2 or SEQ. ID. NO 4 will be a homolog. A percent amino acid sequence similarity or identity is calculated using the methods described herein, preferably using the computer programs described herein. For example, a percent amino acid sequence homology or identity is calculated as the percentage of aligned amino acids that match the reference sequence, where the sequence alignment has been determined using the alignment algorithm of Dayhoff et al; Methods in Enzymology 91: 524-545 (1983).

The invention also contemplates isoforms of the polypeptides of the invention. An isoform contains the same number and kinds of amino acids as the polypeptide of the invention, but the isoform has a different molecular structure. The isoforms contemplated by the present invention are those having the same properties as a polypeptide of the invention as described herein.

The present invention also includes polypeptides of the invention conjugated with a selected polypeptide, or a marker polypeptide (see below) to produce fusion polypeptides. Additionally, immunogenic portions of a polypeptide of the invention are within the scope of the invention.

A polypeptide of the invention may be prepared using recombinant DNA methods. Accordingly, the nucleic acid molecules of the present invention having a sequence which encodes a polypeptide of the invention may be incorporated in a known manner into an appropriate expression vector which ensures good expression of the polypeptide. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a vector of the invention containing a nucleic acid molecule of the invention, and optionally the necessary regulatory sequences for the transcription and translation of the inserted polypeptide-sequence. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. The necessary regulatory sequences may be supplied by a native polypeptide and/or its flanking regions.

The invention further provides a vector comprising a DNA nucleic acid molecule of the invention cloned into the vector in an antisense orientation. That is, the DNA molecule is linked to a regulatory sequence in a manner which allows for expression, by transcription of the DNA molecule, of an RNA molecule which is antisense to a nucleic acid sequence of a nucleic acid molecule of the invention. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The expression vector of the invention may also contain a marker gene which facilitates the selection of host cells transformed or transfected with a vector of the invention. Examples of marker genes are genes encoding a polypeptide such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The vectors may also contain genes which encode a fusion moiety which provides increased expression of the recombinant polypeptide; increased solubility of the recombinant polypeptide; and aid in the purification of the target recombinant polypeptide by acting as a ligand in affinity purification. For example, a proteolytic cleavage site may be added to the target recombinant polypeptide to allow separation of the recombinant polypeptide from the fusion moiety subsequent to purification of the fusion polypeptide. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the recombinant polypeptide.

The vectors may be introduced into host cells to produce a transformed or transfected host cell. The terms “transfected ” and “transfection” encompass the introduction of nucleic acid (e.g. a vector) into a cell by one of many standard techniques. A cell is “transformed” by a nucleic acid when the transfected nucleic acid effects a phenotypic change. Prokaryotic cells can be transfected or transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the polypeptides of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the polypeptide in a desired fashion. Host systems or cell lines may be selected which have specific and characteristic mechanisms for post-translational processing and modification of polypeptides. For example, eukaryotic host cells including CHO, VERO, BHK, HeLA, COS, MDCK, 293, 3T3, and WI38 may be used. For long-term high-yield stable expression of the polypeptide, cell lines and host systems which stably express the gene product may be engineered.

Host cells and in particular cell lines produced using the methods described herein may be particularly useful in screening and evaluating substances or compounds that modulate the activity of a polypeptide of the invention.

The polypeptides of the invention may also be expressed in non-human transgenic animals including but not limited to mice, rats, rabbits, guinea pigs, micro-pigs, goats, sheep, pigs, non-human primates (e.g. baboons, monkeys, and chimpanzees) (see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866). Procedures known in the art may be used to introduce a nucleic acid molecule of the invention encoding a polypeptide of the invention into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries a nucleic acid molecule of the invention in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant polypeptide of the invention in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against Hzf or Hhl Polypeptide.

The polypeptides of the invention may also be prepared by chemical synthesis using techniques well known in the chemistry of polypeptides such as solid phase synthesis (Merrifield, 1964, J. Am. Chem. Assoc. 85:2149-2154), or synthesis in homogenous solution (Houbenweyl, 1987, Methods of Organic Chemistry, ed. E. Wansch, Vol. 15 I and II, Thieme, Stuttgart).

N-terminal or C-terminal fusion or chimeric polypeptides comprising a polypeptide of the invention conjugated with other molecules, such as polypeptides m (e.g. markers) may be prepared by fusing, through recombinant techniques, the N-terminal or C-terminal of a polypeptide of the invention, and the sequence of a selected polypeptide or marker polypeptide with a desired biological function. The resultant fusion polypeptides contain a polypeptide of the invention fused to the selected polypeptide or marker polypeptide as described herein. Examples of polypeptides which may be used to prepare fusion polypeptides include immunoglobulins, glutathione-S-transferase (GST), hemagglutinin (HA), and truncated myc.

Antibodies

A polypeptide of the invention can be used to prepare antibodies specific for the polypeptides. Antibodies can be prepared which bind a distinct epitope in an unconserved region of the polypeptide. An unconserved region of the polypeptide is one that does not have substantial sequence homology to other polypeptides. A region from a conserved region such as a well-characterized sequence can also be used to prepare an antibody to a conserved region of a polypeptide of the invention. Antibodies having specificity for a polypeptide of the invention may also be raised from fusion polypeptides created by expressing fusion polypeptides in host cells as described herein.

The invention can employ intact monoclonal or polyclonal antibodies, and immunologically active fragments (e.g. a Fab or (Fab)₂ fragment), an antibody heavy chain, and antibody light chain, a genetically engineered single chain Fᵥ molecule (Ladner et al, U.S. Pat. No. 4,946,778), humanized antibody, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Applications of the Nucleic Acid Molecules, Polypeptides, and Antibodies of the Invention

The nucleic acid molecules, Hzf or Hhl Polypeptide, or a Hzf or Hhl Related Polypeptide, and antibodies of the invention may be used in the prognostic and diagnostic evaluation of conditions requiring modulation of a nucleic acid or polypeptide of the invention, including disorders of the hematopoietic system, and the identification of subjects with a predisposition to such conditions (See below). Methods for detecting nucleic acid molecules and polypeptides of the invention can be used to monitor such conditions by detecting and localizing the nucleic acids and polypeptides. It would also be apparent to one skilled in the art that the methods described herein may be used to study the developmental expression of the polypeptides of the invention and, accordingly, will provide further insight into the role of the polypeptides. The applications of the present invention also include methods for the identification of substances or compounds that modulate the biological activity of a polypeptide of the invention (See below). The substances, compounds, antibodies etc may be used for the treatment of conditions requiring modulation of polypeptides of the invention including hematopoietic disorders. (See below).

Diagnostic Methods

A variety of methods can be employed for the diagnostic and prognostic evaluation of conditions requiring modulation of a nucleic acid or polypeptide of the invention (e.g. hematopoietic disorders), and the identification of subjects with a predisposition to such conditions. Such methods may, for example, utilize nucleic acid molecules of the invention, and fragments thereof, and antibodies directed against polypeptides of the invention, including peptide fragments. In particular, the nucleic acids and antibodies may be used, for example, for: (1) the detection of the presence of Hzf or Hhl mutations, or the detection of either over-or under-expression of hzf or hhl mRNA relative to a non-disorder state or the qualitative or quantitative detection of alternatively spliced forms of hzf or hhl transcripts which may correlate with certain conditions or susceptibility toward such conditions; or (2) the detection of either an over- or an under-abundance of a polypeptide of the invention relative to a non-disorder state or the presence of a modified (e.g., less than full length) polypeptide of the invention which correlates with a disorder state, or a progression toward a disorder state.

The methods described herein may be performed by utilizing pre-packaged diagnostic kits comprising at least one specific nucleic acid or antibody described herein, which may be conveniently used, e.g., in clinical settings, to screen and diagnose patients and to screen and identify those individuals exhibiting a predisposition to developing a disorder.

Nucleic acid-based detection techniques and peptide detection techniques are described below. The samples that may be analyzed using the methods of the invention include those which are known or suspected to express hzf or hhl or contain a polypeptide of the invention. The methods may be performed on biological samples including but not limited to cells, lysates of cells which have been incubated in cell culture, chromosomes isolated from a cell (e.g. a spread of metaphase chromosomes), genomic DNA (in solutions or bound to a solid support such as for Southern analysis), RNA (in solution or bound to a solid support such as for northern analysis), cDNA (in solution or bound to a solid support), an extract from cells or a tissue, and biological fluids such as serum, urine, blood, and CSF. The samples may be derived from a patient or a culture.

The polypeptides of the invention are primarily expressed in hematopoietic lineages, and in particular in megakaryocytes. The polypeptides of the invention have a role in proliferation, differentiation, activation and/or metabolism of cells of the hematopoietic lineages. Therefore, the methods described herein for detecting nucleic acid molecules and polypeptides can be used to monitor proliferation, differentiation, activation and/or metabolism of cells of the hematopoietic lineage (e.g. megakaryocytes) by detecting and localizing polypeptides and nucleic acid molecules of the invention. The methods described herein may be used to study the developmental expression of a polypeptide of the invention and, accordingly, will provide further insight into the role of the polypeptide in the hematopoietic system.

The nucleic acid molecules and polypeptides of the invention are markers for hematopoietic cells (e.g. megakaryoctyes) and accordingly the antibodies and probes described herein may be used to label these cells.

Methods for Detecting Nucleic Acid Molecules of the Invention

The nucleic acid molecules of the invention allow those skilled in the art to construct nucleotide probes for use in the detection of nucleic acid sequences of the invention in biological materials. Suitable probes include nucleic acid molecules based on nucleic acid sequences encoding at least 5 sequential amino acids from regions of the Hzf or Hhl Polypeptide, or a Hzf or Hhl Related Polypeptide (see SEQ. ID. No. 1 or 3), preferably they comprise 15 to 30 nucleotides. Examples of probes are shown in SEQ. ID. NOs. 5 to 14.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect Hzf or Hhl genes, preferably in human cells. The nucleotide probes may also be useful for example in the diagnosis or prognosis of conditions such as hematopoietic disorders, and in monitoring the progression of these conditions, or monitoring a therapeutic treatment.

The probe may be used in hybridization techniques to detect a hzf or hhl gene. The technique generally involves contacting and incubating nucleic acids (e.g. recombinant DNA molecules, cloned genes) obtained from a sample from a patient or other cellular source with a probe of the present invention under conditions favourable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving hzf or hhl structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations in a hzf or hhl gene. The polymorphisms may be used to identify individuals in families that are likely to carry mutations. If a polymorphism exhibits linkage disequalibrium with mutations in the hzf or hhl gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of hzf or hhl expression. For example, RNA may be isolated from a cell type or tissue known to express hzf or hhl and tested utilizing the hybridization (e.g. standard Northern analyses) or PCR techniques referred to herein. The techniques may be used to detect differences in transcript size that may be due to normal or abnormal alternative splicing. The techniques may be used to detect quantitative differences between levels of full length and/or alternatively splice transcripts detected in normal individuals relative to those individuals exhibiting symptoms of a disease.

The primers and probes may be used in the above-described methods in situ i.e directly on tissue sections (fixed and/or frozen) of patient tissue obtained from biopsies or resections.

Oligonucleotides or longer fragments derived from any of the nucleic acid molecules of the invention may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Methods for Detecting Polypeptides

Antibodies specifically reactive with a Hzf or Hhl Polypeptide, a Hzf or Hhl Related Polypeptide, or derivatives, such as enzyme conjugates or labeled derivatives, may be used to detect Hzf or Hhl Polypeptides or Hzf or Hhl Related Polypeptides in various biological materials. They may be used as diagnostic or prognostic reagents and they may be used to detect abnormalities in the level of Hzf or Hhl Polypeptides or Hzf or Hhl Related Polypeptides, expression, or abnormalities in the structure, and/or temporal, tissue, cellular, or subcellular location of the polypeptides. Antibodies may also be used to screen potentially therapeutic compounds in vitro to determine their effects on a condition such as a hematopoietic disorder. In vitro immunoassays may also be used to assess or monitor the efficacy of particular therapies. The antibodies of the invention may also be used in vitro to determine the level of Hzf or Hhl Polypeptide or Hzf or Hhl Related Polypeptide expression in cells genetically engineered to produce a Hzf or Hhl Polypeptide, or Hzf or Hhl Related Polypeptide.

The antibodies may be used in any known immunoassays that rely on the binding interaction between an antigenic determinant of a polypeptide of the invention, and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify polypeptides of the invention in a sample in order to determine their role in particular cellular events or pathological states, and to diagnose and treat such pathological states.

In particular, the antibodies of the invention may be used in immuno-histochemical analyses, for example, at the cellular and sub-subcellular level, to detect a polypeptide of the invention, to localise it to particular cells and tissues, and to specific subcellular locations, and to quantitate the level of expression.

Cytochemical techniques known in the art for localizing antigens using light and electron microscopy may be used to detect a polypeptide of the invention. Generally, an antibody of the invention may be labeled with a detectable substance and a polypeptide may be localised in tissues and cells based upon the presence of the detectable substance. Various methods of labeling polypeptides are known in the art and may be used. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, β-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies, etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a polypeptide of the invention. By way of example, if the antibody having specificity against a polypeptide of the invention is a rabbit IgG antibody, the second antibody may be goat anti-rabbit gamma-globulin labeled with a detectable substance as described herein.

Where a radioactive label is used as a detectable substance, a polypeptide of the invention may be localized by radioautography. The results of radioautography may be quantitated by determining the density of particles in the radioautographs by various optical methods, or by counting the grains.

Methods for Identifying or Evaluating Substances/Compounds

The methods described herein are designed to identify substances or compounds that modulate the biological activity of a Hzf or HHl Polypeptide or Hzf or HHl Related Polypeptide. “Modulate” refers to a change or an alteration in the biological activity of a polypeptide of the invention. Modulation may be an increase or a decrease in activity, a change in characteristics, or any other change in the biological, functional, or immunological properties of the polypeptide.

Substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. A substance or compound may be an endogenous physiological compound or it may be a natural or synthetic compound.

Substances which modulate a Hzf or Hhl Polypeptide or Hzf or Hhl Related Polypeptide can be identified based on their ability to associate with a Hzf or Hhl Polypeptide or Hzf or Hhl Related Polypeptide. Therefore, the invention also provides methods for identifying substances which associate with a Hzf or Hhl Polypeptide or Hzf or Hhl Related Polypeptide. Substances identified using the methods of the invention may be isolated, cloned and sequenced using conventional techniques. A substance that associates with a polypeptide of the invention may be an agonist or antagonist of the biological or immunological activity of a polypeptide of the invention.

The term “agonist”, refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which can associate with a polypeptide of the invention may be identified by reacting the polypeptide with a test substance which potentially associates with the polypeptide, under conditions which permit the association, and removing and/or detecting the associated polypeptides and substance. Substance-polypeptide complexes, free substance, non-complexed polypeptide, or activated polypeptide may be assayed. Conditions which permit the formation of complexes may be selected having regard to factors such as the nature and amounts of the substance and the polypeptide.

Substance-polypeptide complexes, free substances or non-complexed polypeptides may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the polypeptide or the substance, or labelled polypeptide, or a labelled substance may be utilized. The antibodies, polypeptides, or substances may be labelled with a detectable substance as described above.

A polypeptide, or the substance used in the method of the invention may be insolubilized. For example, the polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a polypeptide of the invention, by assaying for an agonist or antagonist (i.e. enhancer or inhibitor) of the association of the polypeptide with a substance that associates with the polypeptide. The basic method for evaluating if a compound is an agonist or antagonist of the association of a polypeptide of the invention and a substance that associates with the polypeptide, is to prepare a reaction mixture containing the polypeptide and the substance under conditions which permit the formation of substance- polypeptide complexes, in the presence of a test compound. The test compound may be initially added to the mixture, or may be added subsequent to the addition of the polypeptide and substance. Control reaction mixtures without the test compound or with a placebo are also prepared. The formation of complexes is detected and the formation of complexes in the control reaction but not in the reaction mixture indicates that the test compound interferes with the interaction of the polypeptide and substance. The reactions may be carried out in the liquid phase or the polypeptide, substance, or test compound may be immobilized as described herein.

It will be understood that the agonists and antagonists i.e. inhibitors and enhancers that can be assayed using the methods of the invention may act on one or more of the binding sites on the polypeptide or substance including agonist binding sites, competitive antagonist binding sites, non-competitive antagonist binding sites, or allosteric sites.

The invention also makes it possible to screen for antagonists that inhibit the effects of an agonist of the interaction of the polypeptide with a substance which is capable of binding to the polypeptide. Thus, the invention may be used to assay for a compound that competes for the same binding site of the polypeptide.

The reagents suitable for applying the methods of the invention to evaluate compounds that modulate a polypeptide of the invention may be packaged into convenient kits providing the necessary materials packaged into suitable containers. The kits may also include suitable supports useful in performing the methods of the invention.

Compositions and Treatments

The polypeptides, nucleic acid molecules, substances or compounds identified by the methods described herein, antibodies, and antisense nucleic acid molecules of the invention may be used for modulating the biological activity of a polypeptide or nucleic acid molecule of the invention. The polypeptides etc. may have particular application in the treatment of conditions requiring modulation of cells of the hematopoietic lineage i.e. hematopoietic disorders. Treatable disorders include anemia, thrombocytopenia, leukopenia, myelofibrosis, hypoplasia, disseminated intravascular coagulation, immune, thromocytopenic purpura, myelodysplasia, erythrocytopenia, erythrodegenerative disorders, erythroblastopenia, leukoerythroblastosis, erythroclasis, thalassemia, and granulocytopenia. The polypeptides etc. may be used to modulate production of blood cells in situations where a patient has a disease such as AIDS, which has caused alteration in normal blood cell levels, or in clinical settings in which enhancement of hematopoietic populations is desired, such as in conjunction with bone marrow transplant, or in the treatment of aplasia or myelosuppression caused by radiation, chemical treatment, or chemotherapy. The polypeptides, nucleic acids, etc. may also be used to treat patients in which the desired result is the inhibition of a hematopoietic pathway such as for the treatment of myeloproliferative or other proliferative disorders of blood forming organs such as thromocythemias, polycythemias, and leukemias.

The polypeptides, nucleic acids, (e.g. Hzf and hzf), substances, compounds, and antibodies may have specific application in the regulation of megakaryocytopoiesis. Abnormal enhancement of megakaryocytopoiesis leads to thrombocytosis which is implicated in a number of debilitating blood cell disorders, including leukemias and carcinomas. Therefore, the polypeptides, nucleic acids, etc. may be used in the prevention and treatment of polycythemiz vera, megakaryocytic leukemia, ovarian adenocarcinoma, and lupus erythematosus, and in the prevention and treatment of many common diseases where functioning of platelets requires modulation (e.g. inhibition) such as thrombosis, atherosclerosis, hemorrhage, embolism, myocardial infarction, post-myocardial infarction syndrome, and post-cardiac surgery.

Accordingly, the polypeptides, substances, antibodies, and compounds may be formulated into pharmaceutical compositions for adminstration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

After pharmaceutical compositions have been prepared, they can be placed in an appropriate container and labeled for treatment of an indicated condition. For administration of a polypeptide, nucleic acids, etc. of the invention, such labeling would include amount, frequency, and method of administration.

The polypeptides, nucleic acids, etc. and compositions of the invention may be used alone, or in combination with another pharmaceutically active agent such as, for example, cytokines, neurotrophins, interleukins, etc. For example, the polypeptides etc. and compositions may be used in conjunction with any of a number of the above-referenced factors which are known to induce stem cell or other hematopoietic precursor proliferation, or factors acting on later cells in the hematopoietic pathway including but not limited to hemopoietic maturation factor, thrombopoietin, stem cell factor, erythropoietin, G-CSF, GM-CSF, etc.

The invention also contemplates an antibody that specifically binds the therapeutically active ingredient used in a treatment or composition of the invention. The antibody may be used to measure the amount of the therapeutic molecule in a sample taken from a patient for purposes of monitoring the course of therapy.

The nucleic acid molecules encoding a polypeptide of the invention or any fragment thereof, or antisense sequences may be used for therapeutic purposes. Antisense to a nucleic acid molecule encoding a polypeptide of the invention may be used in a situation to block the synthesis of the polypeptide. In particular, cells may be transformed with sequences complementary to nucleic acid molecules encoding a Hzf or Hhl Polypeptide or Hzf or Hhl Related Polypeptide. Thus, antisense sequences may be used to modulate activity or to achieve regulation of gene function. This technology is well known in the art, and sense or antisense oligomers or larger fragments, can be designed from various locations along the coding or regulatory regions of sequences encoding a polypeptide of the invention.

Expression vectors may be derived from retroviruses, adenoviruses, herpes or vaccinia viruses or from various bacterial plasmids for delivery of nucleic acid sequences to the target organ, tissue, or cells. Vectors that express antisense nucleic acid sequences of Hzf or Hhl Polypeptides can be constructed using techniques well known to those skilled in the art.

Genes encoding a Hzf or Hhl Polypeptide can be turned off by transforming a cell or tissue with expression vectors that express high levels of a nucleic acid molecule or fragment thereof which encodes a polypeptide of the invention. Such constructs may be used to introduce untranslatable sense or antisense sequences into a cell. Even if they do not integrate into the DNA, the vectors may continue to transcribe RNA molecules until all copies are disabled by endogenous nucleases. Transient expression may last for extended periods of time (e.g a month or more) with a non-replicating vector, or if appropriate replication elements are part of the vector system.

Modification of gene expression may be achieved by designing antisense molecules, DNA, RNA, or PNA, to the control regions of a hzf or hhl gene i.e. the promoters, enhancers, and introns. Preferably the antisense molecules are oligonucleotides derived from the transcription initiation site (e.g. between positions −10 and +10 from the start site). Inhibition can also be achieved by using triple-helix base-pairing techniques. Triple helix pairing causes inhibition of the ability of the double helix to open sufficiently for the binding of polymerases, transcription factors, or regulatory molecules (see Gee J. E. et al (1994) In: Huber, B. E. and B. I. Carr, Molecular and Immunologic Approaches, Futura Publishing Co., Mt. Kisco, N.Y.). An antisense molecule may also be designed to block translation of mRNA by inhibiting binding of the transcript to the ribosomes.

Ribozymes, enzymatic RNA molecules, may be used to catalyze the specific cleavage of RNA. Ribozyme action involves sequence-specific hybridization of the ribozyme molecule to complementary target RNA, followed by endonucleolytic cleavage. For example, hammerhead motif ribozyme molecules may be engineered that can specifically and efficiently catalyze endonucleolytic cleavage of sequences encoding a polypeptide of the invention.

Specific ribosome cleavage sites within any RNA target may be initially identified by scanning the target molecule for ribozyme cleavage sites which include the following sequences: GUA, GUU, and GUC. Short RNA sequences of between 15 and 20 ribonucleotides corresponding to the region of the cleavage site of the target gene may be evaluated for secondary structural features which may render the oligonucleotide inoperable. The suitability of candidate targets may be evaluated by testing accessibility to hybridization with complementary oligonucleotides using ribonuclease protection assays.

Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The therapeutic index is the dose ratio of therapeutic to toxic effects and it can be expressed as the ED50/LD50 ratio. Pharmaceutical compositions which exhibit large therapeutic indices are preferred.

Other Applications

The nucleic acid molecules disclosed herein may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a polypeptide of the invention. Cells, tissues, and non-human animals lacking in expression or partially lacking in expression of a nucleic acid molecule or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded polypeptide using the methods described herein. Such cells may then be fused to embryonic stem cells to generate transgenic non-human animals deficient in a polypeptide of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a Hzf or Hhl Polypeptide. In an embodiment the invention provides a transgenic non-human mammal all of whose germ cells and somatic cells contain a recombinant expression vector that inactivates or alters a gene encoding a Hzf Polypeptide resulting in a Hzf associated pathology. Further the invention provides a transgenic non-human mammal which does not express or has altered (e.g. reduced) expression of a Hzf or Hhl polypeptide of the invention. In an embodiment, the invention provides a transgenic non-human mammal which does not express or has altered (e.g. reduced) expression of a Hzf polypeptide of the invention resulting in a Hzf associated pathology.

An Hzf associated pathology refers to a phenotype observed for a Hzf homozygous or heterozygous mutant. The phenotype is similar to the phenotype observed after sustained brain damage resulting from stroke in humans. The phenotype of an Hzf homozygous mutant mouse includes the following characteristics: hemorrhage frequently observed in the brain, smaller size, animals are able to lift their heads only to the right or left; and their bodies tremor when placed on unstable surfaces. The membranes of megakaryocytes from the Hzf homozygous mutant animals are also aberrant. Phenotypes observed for Hzf homozygous and heterozygous mutants are also described in the Examples.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with an Hzf or Hhl Polypeptide, preferably a Hzf associated pathology, comprising:

(a) administering the agent to a transgenic non-human animal of the invention; and

(b) determining whether said agent reduces or inhibits the pathology (e.g. Hzf associated pathology) in the transgenic mouse relative to a transgenic mouse of step (a) which has not been administered the agent.

The agent may be useful in the treatment of hematopoietic disorders or disorders requiring modulation of megakaryocytopoiesis, or it may be used to modulate production of blood cells as discussed herein. The agents may be particularly useful in the treatment and prophylaxis of thrombosis, hemorrhage, embolism, stroke, atherosclerosis, myocardial infarction, post-myocardial infarction syndrome, and post-cardiac surgery. The agents may be incorporated in a pharmaceutical composition as described herein.

A polypeptide of the invention may be used to support the survival, growth, migration, and/or differentiation of cells expressing the polypeptide. Thus, a polypeptide of the invention may be used as a supplement to support, for example hematopoietic cells in culture.

The following non-limiting examples are illustrative of the present invention:

### EXAMPLES

### Example 1

Material and Methods

Cells. R1 embryonic stem cells from the 129/Sv strain [34] were maintained on a layer of mitomycin C-treated embryonic fibroblasts in Dulbecco's modified Eagle's culture medium, supplemented with leukemia inhibitory factor, 15% fetal calf serum, L-glutamine, and β-mercaptoethanol as previously described [35]. The OP9 stromal cell line were cultured in α-MEM containing 20% fetal calf serum [33].

Generation of Trapped ES Cell Lines. The plasmid PT1-ATG (PT1 henceforth) contains the En-2 splice acceptor site positioned immediately upstream of the lacZ reporter gene with an ATG translational start site [36]. The bacterial neomycin-resistance (neo) gene is driven by the phosphoglycerate kinase1 (PGK1) promoter. After electroporation of the ES cells with the PT1 vector and selection in G418, drug-resistant clones were plated into 96-well plates. Upon confluency, the cells were expanded into three 96-well plates. One plate was frozen; cells in the second plate were stained with X-galactosidase (X-gal); and cells in the third plate were used for hematopoietic differentiation on OP9 cells.

Analysis of β-galactosidase Activity. β-galactosidase (β-gal) activity was analyzed by staining with 5-bromo 4-chloro 3-indolyl β-galactoside (X-gal; Boehringer Mannheim, Germany). Briefly, cells were washed once with phosphate buffered saline (PBS) and fixed in 0.2% glutaraldehyde, 2 mM MgCl₂ and 5 mM EGTA in 100 mM Na₂HPO₄ for 10 min. at room temperature. Cell cultures were then washed three times for 5 min. each in 100 mM Na₂HPO₄, 0.02% NP-40 and 2 mM MgCl₂ and stained overnight in X-gal staining solution (1 mg/ml X-gal, 5 mM K₃Fe (CN)₆, 5 mM K₄Fe(CN)₆, 2 mM MgCl₂, 0.02% NP-40 in 100 mM Na₂HPO₄) directly in 96- or 6-well plates at 37° C. Following X-gal staining, samples were washed overnight at 40° C. Detection of β-gal activity in embryos was performed as described above except the fixative included 1.5% formaldehyde and fixation was performed for between 30-60 min. depending on the size of the embryo and washed 3 times each for 30 min.

Screening Clones for Hematopoietic Expression. Differentiation of ES cells on OP9 stromal cells was performed essentially as previously described [33]. The drug-resistant clones within each row of a 96-well plate which did not demonstrate X-gal staining in undifferentiated cultures (white clones) were pooled. The pooled clones were seeded onto confluent OP9 cell layers in 6-well plates at a density of 10⁴ cells/well in OP9 media. Cultures were trypsinized at day 5, and 10⁵ cells/well were transferred onto fresh OP9 stroma. Mesoderm-like colonies and hematopoietic cell clusters were observed on day 5 and day 10, respectively. LacZ expression was examined at day 5 and day 10. The pooled clones that demonstrated β-gal activity during the first round of screening were thawed from frozen stocks, and each clone was tested individually for expression of lacZ during hematopoietic differentiation. Clones which demonstrated X-gal staining as undifferentiated cells were analysed individually on OP9 cells.

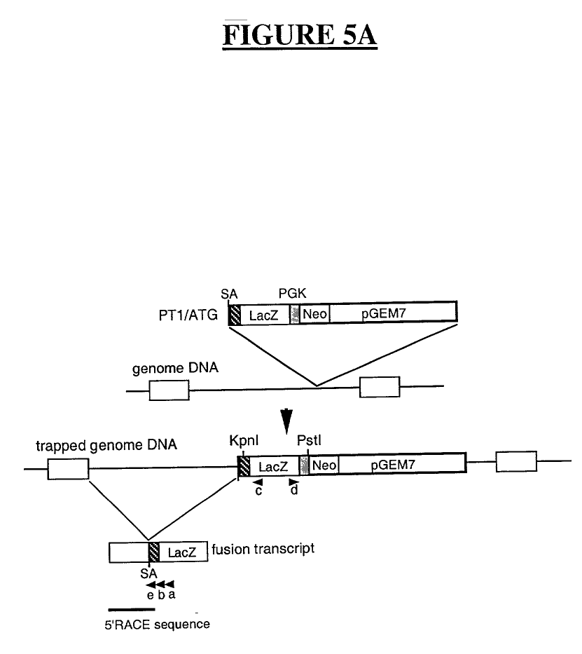

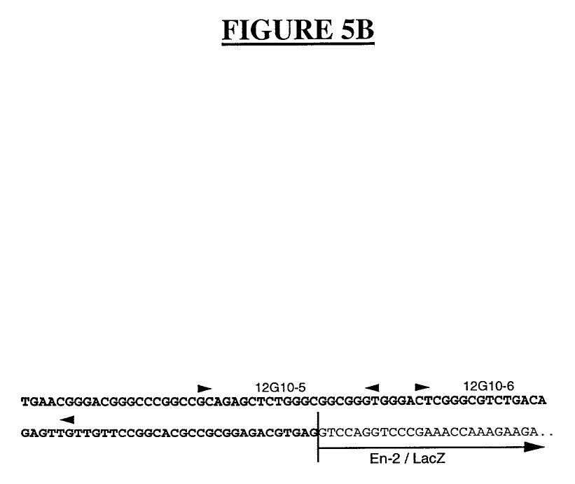

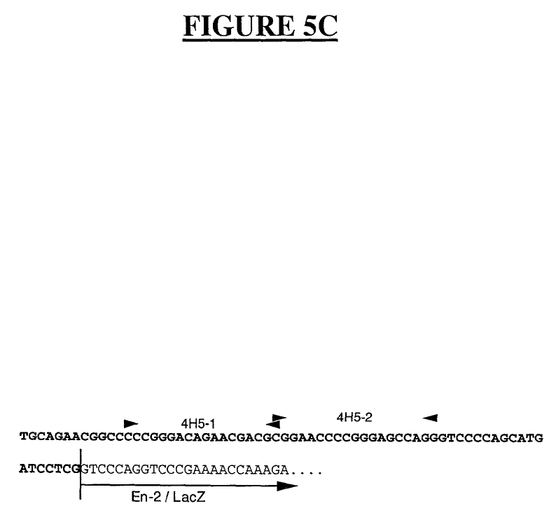

Molecular cloning of the trapped genes by RACE, inverse PCR and cDNA library screening. RNA was prepared from either undifferentiated or differentiated ES cells using Trizol (Gibco/BRL) according to manufacturer's instructions. 5′ RACE was performed using the 5′ RACE kit (Gibco/BRL), according to manufacturer's instructions with modifications previously described17. 5′ RACE products were subcloned into the CloneAmp plasmid (Gibco/BRL) and both strands sequenced using the AutoRead Sequencing kit (Pharmacia) and an A.L.F. DNA Sequencer (Pharmacia). Sequences were analyzed by comparison to the non-redundant GenBank and EST databases of the NCBI using the BLASTN program. 3′ RACE was performed using a 3′ RACE kit (Life Technologies) and two nested primers complimentary to each 5′ RACE fragment (see FIG. 5). Wild type total RNA from day 9 embryos or mouse brain was used as template. Specific primers were as follows: 12G10-5, 5′-GCAGAGCTCTGGGCGGCGGGT-3′; 12G10-6, 5′-CTCGGGCGTCTGACAGAGTTG-3′; 4H5-1, 5′-CCCGGGACAGAACGACGC-3′; 4H5-2, 5′-CGGAACCCCGGGAGCCAGG-3′ (SEQ ID NOs. 5-8, respectively). To obtain a long amplified product, the Expand™ long template PCR system (Boehringer Mannheim) was used. The RACE-PCR products were cloned into the pCR2.1 vector (Invitrogen). The cDNA inserts were sequenced on both strands as previously described. Inverse PCR was performed essential as described by von Melchner et al., (1990) [37]. Genomic DNA from the Hhl ES cell line was digested with PstI and self-ligated at a concentration of 1 μg/ml to obtain circular molecules. 20 ng of self-ligated circular DNA was used for the PCR using the Expand™ long template PCR system (Boehringer Mannheim), with primer sets of GTlacZ-3 [5′-ATTACGCCAGCTGGCGAAAGG-3′] (SEQ.ID.NO.9) and GTlacZ-4 [5′-GATTGGTGGCGACGACTCCTG-3] (SEQ.ID.NO.10). Following digestion with Pst1 and KpnI to isolate flanking sequences just upstream of vector integration sites, the DNA was separated electophoretically on a 0.7% agarose gel and hybridized with a pGEM7 probe.

A 12.5 d.p.c. mouse embryonic cDNA library in the λEXlox vector (mouse strain NIH Swiss; Novagen) was screened by standard procedures [38] using Hhl RACE fragments as probes. Phage plaques (1.0×10⁶) were transferred to nylon filters (Amersham) and hybridized for 2 hr at 68° C. with the radiolabeled probe using Quikhyb solution (Stratagene). Filters were washed at 60° C.: twice for 15 min in 2×SSC and for 1 hour in 0.1×SSC containing 0.1% SDS. Positive clones were plaque purified and the inserts recovered and sequenced as described above. GenBank search and sequence analysis were performed using Fasta, Translate, Bestfit, and Pileup softwares from the Wisconsin Package (Genetics Computer Group, Inc., Madison, Wis.). The ORFs of Hhl and Hzf were submitted to the National Center for Biotechnology Information XREF internet service (http://www.ncbi.nlm.nih.gov/XREFdb/) where the sequences were compared to the most recent updates of the EST database. In addition, the sequences were compared to all public databases using the BLAST algorithm. Generation of Chimeras. The ES cell clones, Hhl and Hzf were aggregated with CD1 diploid embryos as previously described [34] and transferred to foster mothers to generate several strong male chimeras. Chimeric males were bred to CD1 females and tail DNA of F1 and F2 offspring was analyzed by southern blotting and hybridization to En-2/LacZ and RACE fragment probes. For the Hhl clone, genotyping was performed using densitometry, comparing the intensity of the LacZ band to the internal standard, En-2. The reliability of genotyping by quantitative Southern blot analysis was confirmed by test breeding [29]. For the Hzf line, EcoRI-digested DNA was hybridized with the RACE probe which detected a polymorphism between the wild-type and mutant alleles. All the analyses described were performed in the CD-1/129Sv hybrid background. RNA Analysis. Northern blot analysis was carried out according to standard procedures [38]. RT-PCR was performed using an RNA PCR kit (Perkin-Elmer Cetus) using total RNA from the brains of F₂ mice as template. Combinations of specific primers were as follows: 12G10-6; 12G10-14, 5′-TATCTTCAGCTGTGGCTTCCC-3′ (SEQ.ID.NO.11); GT-lacZ-2A 5′-CCGTCGACTC TGGCGCCGCTGCTCTGTCAG-3′ (SEQ.ID.NO.12); for amplification of Hhl products and 4H5-2, 5′-CGGAACCCCGGGAGCCAGG-3′ (SEQ.ID.NO.13); 4H5-6, 5′-TCTGGGGATCCTGGAGCTGGAC-3′ (SEQ.ID.NO.14); GT-lacZ-2A for Hzf products. Immunofluorescence. Cells were stained with the fluorescent β-gal substrate, fluorescein di-β-D-galactopyranoside (FDG) as previously described [39]. Cells (10⁶) were washed twice with phosphate buffered saline (PBS) and resuspended in 40 μl PBS containing 5% FCS. After 10 min of preincubation at 37° C., cells were incubated with 40 μl prewarmed FDG (2 mM in water) for 75 seconds at 37° C. FDG loading was terminated by adding 720 μl ice cold PBS containing 5% FCS. Cells were cytospun onto glass slides and examined under a fluorescent microscope, prior to morphological examination by Wright-Giemsa staining. Cell surface expression of CD61 was performed by indirect immunofluorescence. Bone marrow cells were washed in PBS and incubated with anti-CD61 (PharMingen, ON, Calif.) for 30 minutes, washed twice, then incubated for 30 minutes with fluorescein isothiocyanate (FITC) conjugated mouse anti-hamster IgG monoclonal antibody (PharMingen, ON, Calif.). Cells were washed twice and analysis was performed using a FACS cell sorter (Becton and Dickinson, Mountain View, Calif.) CFU-C Analysis. Bone marrow cells were cultured in methylcellulose containing the growth factors IL-3, IL-6, Epo and SCF (METHOCULT GF M3434; Stemcell Technologies). After 7-16 days, individual colonies were picked and stained with X-gal in a 96-well plate. Colony types were determined morphologically using Wright-Giemsa staining. For analysis of CFU-Mk, bone marrow cells were plated in 0.3% MegaCult serum-free base agarose (StemCell Technologies HCC-4701K) in the presence of recombinant murine IL-3 (20 ng/ml), TPO (40 ng/ml) and SLF (50 ng/ml) [40] and cultured for 18-21 days. The complete culture was stained with X-gal and CFU-Mk were examined microscopically.

Results

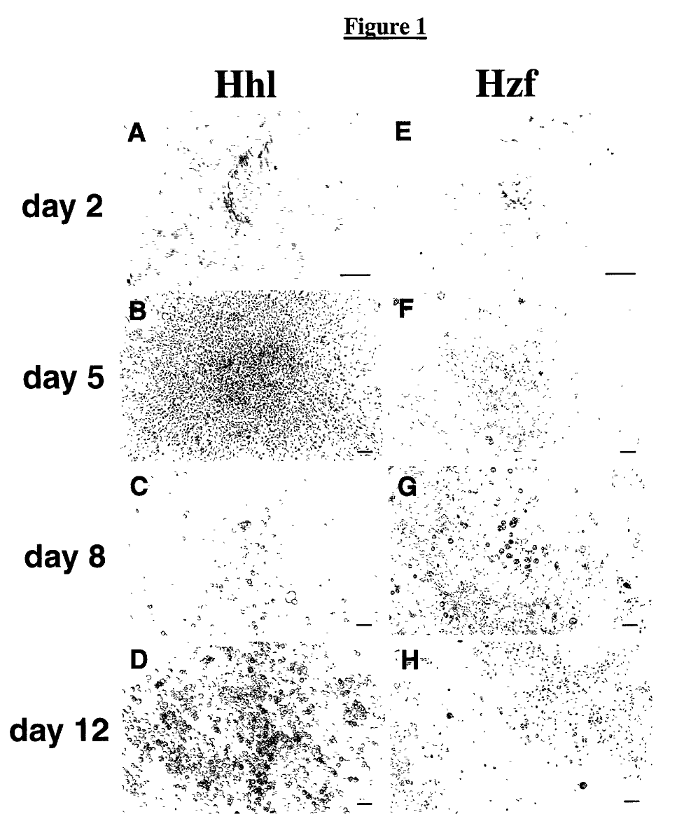

Isolation of hematopoietic gene trap clones. The PT1 gene trap vector, which contains a splice acceptor site immediately upstream of a promoterless lacZ reporter gene and the neo gene driven by the PGK-1 promoter, was introduced into ES cells (clone R1) by electroporation. After selection of neo^{R} colonies, clones were replica plated for freezing, analysis of lacZ expression by undifferentiated ES cells, and differentiation on OP9 stromal cells. Of 1,350 neoR ES clones, 95% did not express lacZ (white clones) and 5% were lacZ positive (blue clones) in the undifferentiated state. Both white and blue clones were allowed to differentiate on OP9 cells and lacZ expression was monitored throughout hematopoietic differentiation. Two of the originally white clones (0.2% of the total number of clones) now expressed lacZ upon hematopoietic differentiation (Table 1). One of these clones, 7H7, expressed lacZ at day in mesoderm-like colonies which was subsequently downregulated during differentiation and not detected in hematopoietic cells by day 10 of culture. The other clone, Hhl, expressed lacZ at both the mesodermal and hematopoietic stages of differentiation (FIG. 1). Approximately one-third of the hematopoietic clusters derived from Hhl ES cells expressed lacZ 12 days after intitiation of co-culture.

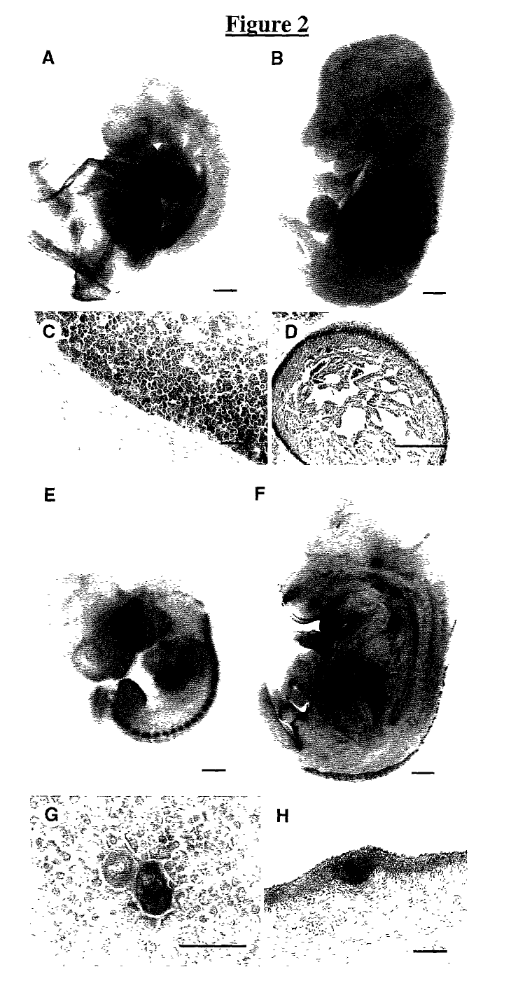

Of the 70 clones that expressed lacZ in the undifferentiated state (Table 1), 97% (68/70) downregulated lacZ expression upon differentiation while two of the originally blue clones, Hzf and 4A11, expressed lacZ throughout the differentiation into hematopoietic cells (Table 1). Clone Hzf expressed lacZ in only a few cells at the undifferentiated (day 2) and mesodermal stages (day 5). Interestingly, lacZ expression in Hzf was restricted to large cells located at the periphery of day 12 hematopoietic clusters (FIG. 1). Clone 4A11 was subsequently shown to be ubiquitously expressed in vivo and was not characterized further. The two hematopoietic regulated gene trap clones Hzf and Hhl are described below. In vivo Expression Analysis. Previously it has been shown that the in vitro patterns of lacZ expression in trapped ES clones accurately predicts the expression patterns observed in vivo [18]. To determine whether the in vitro expression patterns of the trapped genes in the ES clones Hhl and Hzf were also recapitulated in vivo, chimeric mice were generated between these ES clones and diploid embryos and the trapped alleles were transmitted through the germ line to produce F₁ heterozygotes. In Hhl heterozygotes (Hhl/+), lacZ was expressed in the yolk sac and heart primordium at 8.5 days postcoitum (d.p.c) and in the heart, dorsal root ganglia and fetal liver after 12.5 d.p.c (FIG. 2A & B). Virtually all cells in 14.5 d.p.c fetal liver were positive for X-gal staining (FIG. 2C), and lacZ expression in the heart was localized to the endocardium and myocardium (FIG. 2D). In Hzf heterozygotes (Hzf/+), lacZ was expressed in the somites, basal ganglia, apical ectodermal region of the limb buds and liver primordium in 9.5 d.p.c. embryos (FIG. 2E). Expression was also observed in the skin (FIG. 2H), trigeminal ganglia, thymus, salivary gland and spinal cord, with punctate staining in the fetal liver around 14.5 d.p.c (FIG. 2F). The punctate staining in the liver was due to the restricted staining of large polynuclear cells resembling megakaryocytes (FIG. 2G).

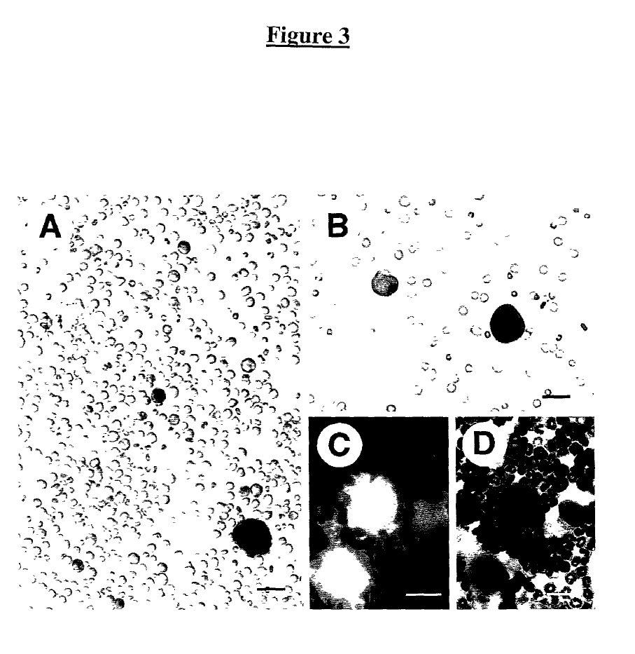

To characterize further the expression pattern of the trapped genes within the hematopoietic compartment, bone marrow cells were isolated from adult mice and analyzed for lacZ expression. Unfractionated cells obtained from Hzf/+ mice expressed high levels of lacZ in about 2% of total bone marrow cells while up to 80% (ranging from very high to low levels of expression) of cells obtained from Hhl/+ mice were lacZ-positive (for example, see FIGS. 3A & B). Both lines demonstrated a coincidence of lacZ expression, detected by FDG staining, within cells morphologically resembling megakaryocytes (FIGS. 3C & D).

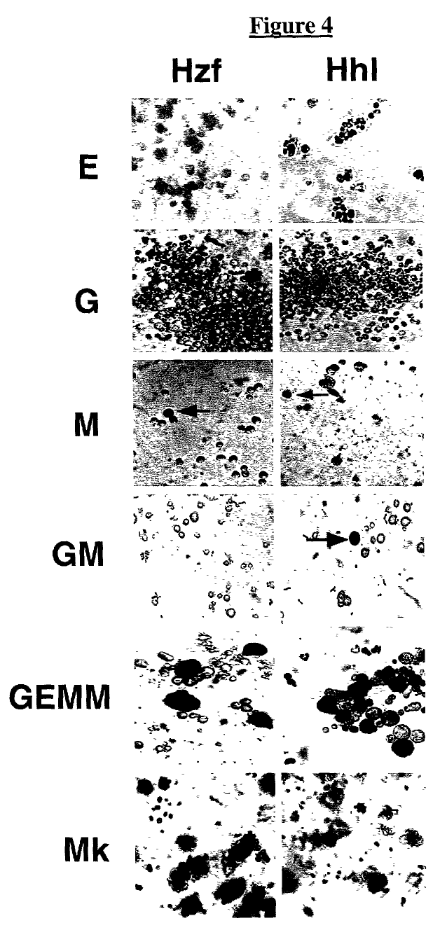

This pattern of lacZ expression was further supported by the expression of lacZ within most megakaryocyte colony forming units (CFU-Mk) generated from Hhl/+ and Hzf/+ bone marrow in a serum-free agarose assay in the presence of TPO, IL-3 and SLF (FIG. 4). In addition to expression in megakaryocytes, lacZ was expressed in a population of cells within CFU-M and CFU-GEMM colonies, with no expression observed in CFU-GM, G or BFU-E colonies derived from Hzf/+ bone marrow cells (FIG. 4). In contrast, lacZ was expressed to high levels in BFU-E and cells within CFU-GEMM colonies from Hhl/+ bone marrow cells. Expression was also observed in some cells within CFU-M and the macrophage component of CFU-GM (FIG. 4); however, granulocytes did not express lacZ. Thus, the in vitro hematopoietic expression of both Hhl and Hzf was recapitulated in vivo.

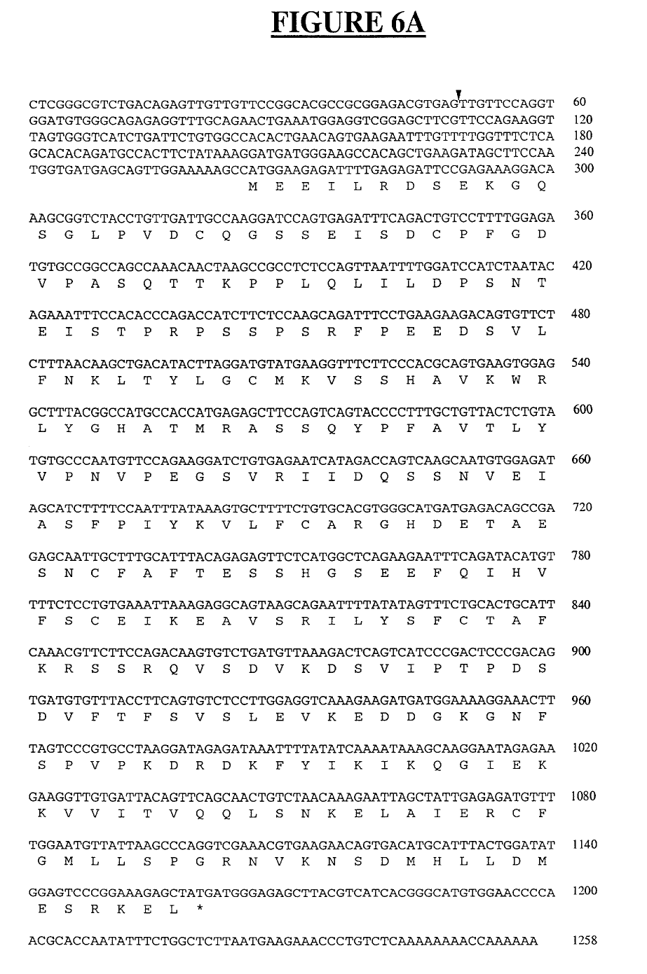

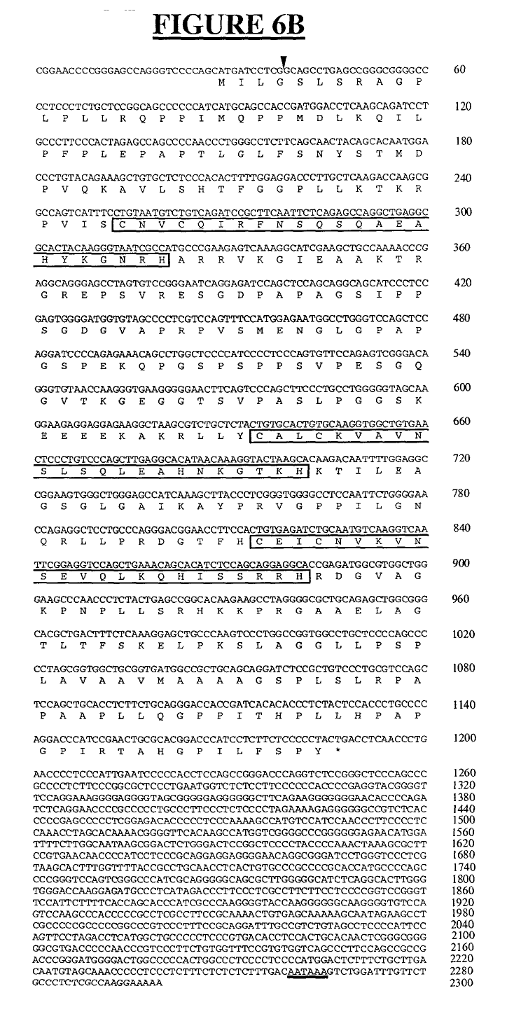

To define more precisely the cells that expressed lacZ, bone marrow cells were selected on the basis of their expression of the megakaryocyte cell surface marker, CD61, by fluorescence-activated cell sorting. Approximately 5% of bone marrow cells derived from Hzf/+ mice expressed lacZ. About 0.5-1% of the lacZ-positive cells did not express CD61. Within the CD61+ fraction obtained from Hhl/+ bone marrow cells, approximately 14% of the cells expressed lacZ whereas 33% demonstrated lacZ expression in the CD61- fraction. Sequence analysis of Hzf and Hhl. To determine the primary sequence of the Hzf and Hhl trapped genes, lacZ fusion transcripts were cloned by 5′ rapid amplification of cDNA ends (RACE; FIG. 5). Primers corresponding to the sequence obtained by 5′ RACE were used for subsequent 3′-RACE to obtain further sequence downstream of the vector integration site (FIG. 5 & 6). The full length coding sequence for Hzf was obtained by 3′ RACE (FIG. 6B.). A combination of RACE, inverse PCR and cDNA library screening was used to obtain partial sequences of Hhl with a full length coding sequence being obtained using 3′ RACE (FIG. 6A). Hhl. The Hhl sequence is 1,258 base pairs (bp) in length and contains an uninterrupted open reading frame (ORF) which encodes a putative polypeptide of 298 amino acids (FIG. 6A). The sequence of this ORF does not share significant similarity with any known genes; however, the 3′ end of Hhl shares 88% DNA similarity over 126 bases to a murine expressed sequence tag (EST) isolated from a heart cDNA library (accession # AA919544) using the NCBI search program. Analysis of the translated sequence demonstrated significant homology (33.47%, determined by using Bestfit and GAP programs from GCG) to a peptide encoded by a series of C.elegans cDNAs (accession # U41540) using Psi blast from the NCBI search program as well as FASTA3 from the ExPasy Program. The cloned Hhl cDNA may not contain the full-length message because it contains no classical AATAAA polyadenylation signal apparent in its 3′ UTR.

Analysis of the translated sequence using ProfileScan from the ExPasy Program identified a putative phosphotyrosine binding domain between amino acids 78-192.

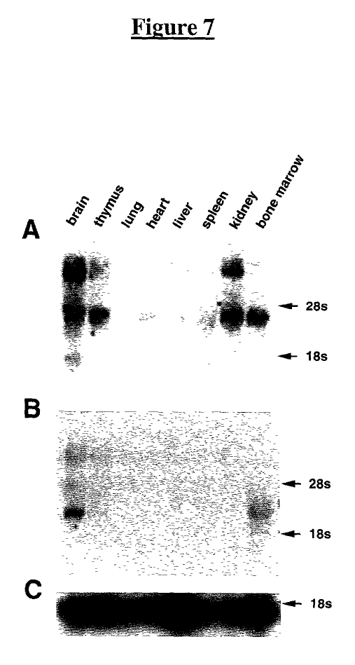

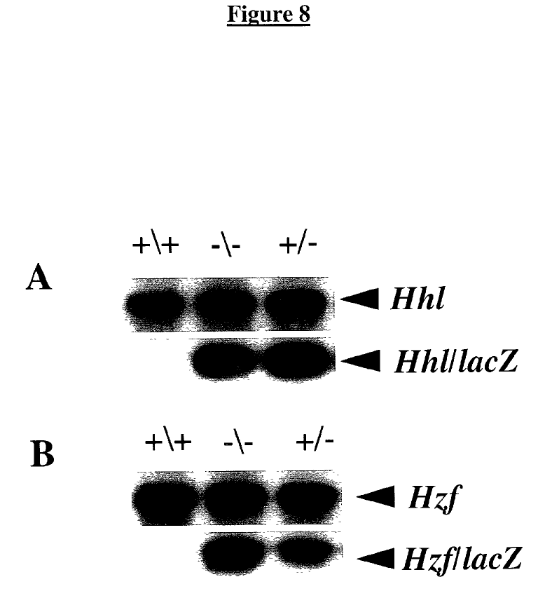

Hzf The Hzf sequence is 2,300 bp in length and contains a 396 amino acid ORF with a putative translation start site at nucleotide 58 embedded in a consensus Kozak sequence. There is a single AATAAA polyadenylation signal in the 3′UTR. Sequence analysis demonstrated that Hzf is a novel gene encoding a polypeptide that includes 3 zinc finger motifs of the C₂H₂ type (FIG. 6B). The Hzf putative peptide has sequence similarity (22-32%) with several C₂H₂ type zinc finger containing polypeptides, including the Xenopus laevis dsRBP-Zfa (41), the murine p53-inducible zinc finger polypeptide, Wig-1 (42) and its rat homolog PAG608 polypeptide (43). RNA in situ hybridization and northern analysis. To determine whether the expression pattern of lacZ described above reflected the endogenous expression pattern of the trapped genes, the X-gal staining pattern in 14.5 d.p.c heterozygous embryos was compared with the pattern of expression observed by RNA in situ hybridization analysis in wild type 14.5 d.p.c embryos. In general, the RNA in situ signals for both Hzf and Hhl coincided with the patterns observed by staining for lacZ expression (data not shown). Northern blot analysis revealed Hhl transcripts of 3 different sizes in various tissues, suggesting that Hhl undergoes differential splicing in a tissue-specific manner in adult tissues (FIG. 7A). A single 2.3kb Hzf message was observed in the bone marrow and brains of adult mice and at lower levels in the thymus (FIG. 7B). Mice homozygous for the Hzf and Hhl gene trap insertion are viable and fertile. Hzf and Hhl heterozygous mice do not exhibit any apparent abnormalities and are fertile. Intercrosses of F1 heterozygous mice for each of the Hhl and Hzf lines generated viable homozygous offspring at the appropriate Mendelian ratios. Mice homozygous for either insertion developed normally and did not exhibit any overt phenotype, including the numbers of types of hematopoietic progenitor cells, as determined by in vitro colony assays (data not shown). The absence of a discernible phenotype in these mice might reflect the absence of any effect on gene expression as the result of the integration of the PT1 vector into the Hhl and Hzf genes. To test this possibility, expression of both Hzf and Hhl RNA transcripts was analyzed in the brains of wild type and homozygous mice by RT-PCR using primers that span the integration site. For both Hzf and Hhl, cDNA was amplified from both Hzf and Hhl homozygotes, suggesting that transcripts that extend 3′ to the integration site were present (FIG. 8). This conclusion was further confirmed by Northern blot analysis using probes 3′ to the site of vector integration. This analysis demonstrated no differences in the levels or size of the transcripts obtained from wild-type or homozygous mice. As shown in FIG. 6A, vector integration within the Hhl locus was within the 5′ UTR upstream of the first coding exon. For Hzf, vector integration occurred downstream of the translational start site but upstream of an additional start site which also has a good Kozak consensus; therefore, alternative initiation codon usage may have occurred (FIG. 6B). Thus, the sites of vector integration could account for the lack of mutagenesis of these trapped loci. Alternatively, splicing around the gene trap vector may have occurred which would also result in the expression of endogenous transcript. The latter has been reported to occur for a number of trapped genes [44, 45, 46, 47, 48, 49] and splicing around targeting constructs has also been shown to occur, resulting in the generation of partially functional polypeptides rather than the expected null mutation [50].

Discussion

Novel genes regulated during hematopoietic development were identified and characterized. ES cell clones containing a random gene trap insertion were induced to differentiate into hematopoietic cells by co-culture on OP9 stromal cells. Clones were screened for lacZ expression in undifferentiated cells as well as at various stages during hematopoietic differentiation. From a total of 1,350 ES clones, three clones exhibited lacZ expression within hematopoietic cells and two of these clones displayed a regulated expression pattern in vitro which was recapitulated during hematopoietic development in vivo. Molecular cloning and cDNA sequence analysis of the trapped genes in these two ES clones revealed that both genes, which have been denoted as Hzf and Hhl, are novel. Thus, this in vitro expression-based gene trap strategy is a successful approach for screening for novel hematopoietic genes.

In undifferentiated Hzf ES cells, lacZ expression was observed in only a few cells and this low frequency of expression was maintained throughout hematopoietic differentiation in vitro. The expression of Hhl was upregulated being first detected in mesodermal colonies and later expressed in the majority of hematopoietic cells by day 12. During embryogenesis, neither Hzf or Hhl was expressed in hematopoietic cells within blood islands in the extraembryonic yolk sac, the site of primitive hematopoiesis. Neither gene was expressed within the intraembryonic para-aortic splanchnopleur/aorta-gonad-mesonephros (p-Sp/AGM) region, the site where definitive hematopoiesis is thought to be initiated [51, 52]. This lack of detectable Hhl or Hzf expression in these hematopoietic compartments does not exclude the possibility that cells within these regions express these genes. It is possible that in these compartments only a low frequency of cells express Hzf or Hhl and/or expression in cells within these regions is below the level of detection. Expression of both trapped genes in hematopoietic cells was observed first in the fetal liver and subsequently adult bone marrow. The low frequency of Hzf expression observed in vitro was recapitulated in vivo in the embryonic liver and in only 2% of adult bone marrow cells. The higher frequency of Hhl expression within day 12 hematopoietic cells in vitro was also recapitulated in vivo where 80% of adult bone marrow cells expressed lacZ. The majority of cells expressing Hzf were megakaryocytes, as determined by morphology, lacZ expression within CFU-Mk, and cell surface expression of the megakaryocyte-specific surface marker, CD61. Hzf was also expressed in other hematopoietic cell lineages contained within CFU-GEMM and at a very low frequency in CFU-M. Hhl was more widely expressed than Hzf within hematopoietic lineages, particularly in erythroid and megakaryocyte cells as well as in the majority of cells comprising CFU-GEMM.

Both Hzf and Hhl were expressed in megakaryocytes, suggesting that these novel genes may play a role in the differentiation and/or maturation of this cell lineage. The major determinants of commitment to the megakaryocyte lineage and the regulation of gene expression in this lineage remain unclear. The generation of several knock-out mice have aided in our understanding of megakaryocyte differentiation. Mice lacking thrombopoietin (TPO) or its receptor c-Mpl have reduced numbers of megakaryocytes and as a consequence are also thrombocytopenic [8, 53]. These mice also have reduced numbers of hematopoietic progenitors of multiple lineages [54, 55]. Mice with a megakaryocyte-selective loss of GATA-1 and NF-E2 null mice are also thrombocytopenic as a result of arrested megakaryocyte differentiation and maturation [56, 57]. Mice lacking GATA-1 or NF-E2 also display defects in erythropoiesis [58, 57], suggesting that these transcription factors are co-expressed within a bipotential stem cell capable of giving rise to both megakaryocytes and erythroid cells. Recently, it has been shown that the multiple zinc finger polypeptide, Friend of GATA-1 (FOG), cooperates with GATA-1 to promote erythroid and megakaryocyte differentiation [59]. In mice lacking FOG due to a targeted null mutation in ES cells, the differentiation of erythroid precursors is blocked [60]. However, in contrast to GATA-1 deficient mice, megakaryocytes are completely absent, suggesting a GATA-1 independent role for FOG perhaps at the very earliest stage of megakaryocyte differentiation from a bipotential erythroid/megakaryocyte progenitor. In this study, Hzf expression was observed in megakaryocytes but not in erythroid cells, suggesting that the function of this novel gene may be restricted to the megakaryocyte lineage. The presence of three zinc-finger motifs in Hzf suggests that it may also be involved in transcriptional regulation in megakaryocytes. In contrast, Hhl was strongly expressed in both erythroid and megakaryocyte lineages. Given that both Hzf and Hhl were also expressed within multipotential CFU-GEMM colonies, these novel genes may be expressed in a bi/multipotential progenitor.

In addition to expression in hematopoietic cells, Hhl was also expressed in the embryonic and adult heart and Hhl has significant homology to an EST isolated from a heart cDNA library. Northern blot analysis of adult mouse tissue also demonstrated Hhl transcripts in the brain and kidney. In adult tissue, Hzf was also expressed in the brain. Thus, these genes may also be involved in other developmental processes outside of the hematopoietic system.

The gene trap strategy resulted in the identification of two novel genes with coincidence of expression within megakaryocytes. Insertion of the gene trap vector into Hzf and Hhl has allowed the molecular cloning of both genes and has also facilitated the rapid analysis of their expression during mouse embryogenesis.

### Example 2

Mice with true null alleles for Hzf were generated. A targeting vector was designed to replace a 5.5 kb genomic fragment containing exons encoding 3 zinc finger domains with a IRES LacZ and a neomycin resistance gene. The IRES LacZ and Neo resistance gene was inserted in the targeting vector in the sense orientation to the Hzf transcript. The targeting vector was electroporated into R1 ES cells. After positive and negative selection, 6 independent clones were screened after checking for homologous recombination using 5 prime and 3 prime flanking probes. A Neo probe was also used to check single integration. Three out of 6 clones were aggregated, resulting in germline transmission. Heterozygous offspring were interbred to generate homozygotes. The total number of new born pups decreased at 3 weeks after birth. In order to determine the stage of neonatal development affected by the Hzf mutation, neonatal genotyping was performed. One week old Hzf−/− pups were viable, usually of normal size and occurred at the expected Mendelian frequency. However, thereafter, typically from 2 to 3 weeks after birth, the percentage of homozygotes was decreased. Most of the Hzf−/− showed behavioral abnormalities. Six out of 24 Hzf−/− mice were able to lift their heads only to the left side. Seventeen out of 24 exhibited tremoring of their bodies when they were put on unstable surfaces (e.g. lid of the cage). Surviving mice were smaller than their littermates. Hemorrhage was frequently observed (e.g. at 3 weeks) in the brain of Hzf homozygous mutants. Therefore, in summary the following phenotype was observed:

(a) The frequency of viable Hzf−/− mice decreased at the age of two weeks, although normal Mendelian ratios of Hzf mutants was observed at one week after birth.

(b) Hemorrhage was frequently observed in the brain of Hfz−/− mice.

(c) The majority of surviving Hzf mice were smaller than their littermates, presumably as a result of sustained hemorrhage during a period of rapid growth; they were able to lift their heads only to the right or left; and their bodies tremor when placed on unstable surfaces. The phenotype resembled the phenotype in the case of sustained brain damage resulting from a stroke in humans.

The ultrastructure of mature megakaryocytes from the Hzf−/− mouse was compared to mature megakaryocytes from the bone marrow of a Hzf+/+ mouse. The cytoplasm of megakaryocytes from the Hzf+/+ mouse showed platelet fields or territories that are clearly demarcated and many granules were observed. In contrast, the cytoplasm of megakaryocytes of the Hzf−/− mouse showed that the organization of the demarcation membrane system is aberrant, and granules are sparse.

### Example 3

In higher vertebrates, anucleate platelets are generated from megakaryocytes and have an essential role in maintaining hemostasis in vivo. During megakaryocyte differentiation, megakaryocyte-restricted progenitor cells commence an unusual process, terminal endomitosis, in which DNA replication occurs but neither the nucleus nor the cell undergoes division (Zuker-Franklin, 1989). Hence, mature megakaryocytes are invariably polyploid and contain 4N to 64N of the normal diploid amount of DNA (Jackson et al., 1984). Because platelets can produce only limited amounts of protein, their cytoplasmic structures are mostly derived from megakaryocytes (Burstein, 1995). Morphologically, four distinct categories of granules differing in their internal constituents are produced by maturing megakaryocytes: (α-granules, dense granules, lysosomal granules, and microperoxisomal granules (Burstein, 1995). The α-granules, which are the most numerous, contain various proteins, including platelet derived growth factor (PDGF), platelet factor (PF) 4, and von Willebrand factor (vWF). These proteins are synthesized in megakaryocytes and then transported to α-granules. These packed α-granules are subsequently shed from megakaryocytes (Greenberg et al., 1987). Thus, the process of megakaryocyte differentiation involves a complex series of cellular processes to generate functionally mature platelets.

Genetic approaches involving gene targeting in mice have revealed several genes and their protein products that are essential for megakaryopoiesis. These include various transcription factors, such as p45 NF-E2 (Shivdasani et al., 1995), mafG (Shavit et al., 1998), GATA-1 (Shivdasani et al., 1997), and Fli-1 (Hart et al., 2000). The transcription factor NF-E2 is a heterodimer of two proteins, p45 and p18, that are members of the CNC and Maf subfamilies, respectively (Blank and Andrews, 1997; Motohashi et al., 1997). Deficiency of the p45 NF-E2 gene blocks completion of megakaryopoiesis and results in the complete absence of platelets (Shivdasani et al., 1995), whereas the absence of MafG leads to hyperproliferation of megakaryocytes and mild thrombocytopenia (Shavit et al., 1998). Mice carrying megakaryocyte-specific deletion of the GATA-1 zinc finger protein exhibit severely impaired cytoplasmic maturation of megakaryocytes and reduced platelet numbers (Shivdasani et al., 1997). The Fli-1 proto-oncogene is a member of the ETS family of winged helix-turn-helix transcription factors that bind a purine-rich consensus sequence GGA(A/T) (Nye et al., 1992; Zhang et al., 1995; Zhang et al., 1993). Mice carrying a null mutation in the Fli-1 gene exhibit a block in megakaryocyte differentiation (Hart et al., 2000).

The process of megakaryocyte differentiation also requires the interaction between thrombopoietin (TPO), also known as megakaryocyte growth and development factor (MGDF), and its cognate receptor c-Mpl (Bartley et al., 1994; de Sauvage et al., 1994; Kato et al., 1995; Kaushansky et al., 1994; Kuter et al., 1994; Lok et al., 1994; Souyri et al., 1990; Wendling et al., 1994). The ligand TPO/MGDF increases megakaryocyte ploidy at low concentrations while the final stage of platelet release is not dependent on TPO/MGDF (Choi et al., 1996). c-mpl-deficient mice exhibit thrombocytopenia and impaired megakaryopoiesis (Alexander et al., 1996; Gurney et al., 1994). In addition, TPO/MGDF null mutant mice have decreased number of both platelets and megakaryocyte progenitors, and lower ploidy levels of megakaryocytes (de Sauvage et al., 1996). Interestingly, both c-Mpl and TPO/MGDF null mice do not suffer a bleeding disorder, suggesting that the platelets produced from the abnormal megakaryocytes might have a normal function.

To investigate the in vivo role of Hzf, mice were generated with a null Hzf mutation by gene targeting. Below the essential function of Hzf in α-granule formation and megakaryopoiesis is described.

The following experimental procedures were used in the study described in this

Example 3:

Generation of Hzf^{-/-} Mutant Mice

DNA fragments corresponding to the murine Hzf gene fragments were cloned from a 129/Sv genomic DNA library using a mouse Hzf cDNA probe (Hidaka et al., 2000). Seventeen overlapping phage genomic clones containing exons encoding three zinc finger domains were isolated. A targeting vector was designed to replace a 5.5 Kb genomic fragment including the exons encoding three zinc finger domains with an internal ribosome-entry site (IRES) LacZ and a neomycin resistant gene (neo). IRES LacZ and neo were inserted in the pPNT targeting vector in the sense orientation to Hzf transcription, such that IRES LacZ was flanked on the 3′ side by 1.6 Kb of genomic DNA and that neo was flanked on the 5′ side by 4.5 Kb of genomic DNA. The targeting vector was linearized with NotI and electroporated into R1 ES cells. After positive-negative selection with gancyclovir and G418, 800 surviving clones were picked and screened by Southern blot analysis. Three out of six homologous recombinant ES clones were aggregated into CD1 blastomeres and transferred to foster mothers to generate chimeras. Chimeric mice were mated with CD1 females, and germline transmission of the mutant allele was verified by PCR and Southern blot analysis of ear punched tissues and tail DNA from F1 offspring. A primer “a” and “b” specific for the Hzf gene (5′-GGACCCTGTACAGAAAGCTGT-3 [SEQ ID. NO. 15] and 5′-GCTTGGTCTACAGAGTGATT-3′ [SEQ ID. NO. 16], respectively) and a primer “c” specific for the IRES gene (5′-GGAGGGAGAGGGGCGGAATT-3′ [SEQ ID. NO. 17]) were used in PCR analysis. Germline transmission of the targeted Hzf allele was achieved for three independent ES clones. F2 offspring from heterozygous intercrosses were genotyped by Southern blotting. Mutant mice derived from three targeted ES cells showed the identical phenotype.

Histological Analysis

Organs were isolated in ice-cold phosphate buffered saline (PBS) 3 weeks after birth, fixed overnight in 4% paraformaldehyde at 4° C. (for brains, fixed at least 1 week in 4% paraformaldehyde at 4° C.), dehydrated, and embedded in paraffin. Sections 5 μm thick were cut and stained with hematoxylin and eosin.

Hematological Analysis

Four week-old mice were anesthetized with methoxyflurane. Peripheral blood was collected by heparinized capillary puncture of the retroorbital venous plexus into a tube containing 5 μl of 0.5 M EDTA, pH 8.0 (Becton Dickinson, Franklin Lakes, N.J.). Peripheral blood cell counts were determined with MASCOT automated hematology system (CDC Technologies, Oxford, Conn.). Blood smears and bone marrow smears were stained with Wright-Giemsa stain.

The preparation of platelets was carried out essentially as described previously (Nieswandt et al., 1999). Peripheral blood, which was prepared as described above, was centrifuged at 300×g for 10 min at room temperature to obtain platelet-rich plasma (PRP). The platelets were washed three times with PBS by centrifugation at 1,300×g for 10 min and were used immediately for Western immunoblot analysis.

Bleeding Time Assays

The bleeding time was measured as described (Dejana et al., 1979; Law et al., 1999) using 4-week-old mice. Two mm of the tip at the tail was cut with a sharp scalpel blade, the tail was placed in a solution of saline at 37° C. and the time for the flow of blood to cease was recorded. Genotyping of the animals took place after this procedure so that tails were intact for bleeding-time measurements. If bleeding restarted within 1 min, this result was recorded as a rebleed and taken to indicate an unstable hemostatic event.

Measurements of Megakaryocyte Frequency and DNA Content

DNA distribution of megakaryocytes in unfractionated bone marrow cells was determined as described previously (Arnold et al., 1997; Jackson et al., 1984). Bone marrow cells were harvested from Hzf mutant and control mice.

CFU-Mk Assay

Megakaryocyte progenitor cells, in the bone marrow from Hzf mutant and control mice, were assayed in collagen-based media (StemCell Technologies, Vancouver, Canada) supplemented with recombinant human TPO (50 ng/ml), IL-6 (20 ng/ml), and IL-11 (50 ng/ml) and recombinant mouse IL-3 (10 ng/ml). Colonies were stained for AChE activity counted after 6 days incubation.

Ultrastructural Studies

Freshly excised spleens obtained from 3- to 5-week-old Hzf^{-/-} and control mice were fixed in 2% gultaraldehyde (Canemco Inc., Dorval, Canada) in 0.1 M sodium cacodylate buffer, pH 7.3 (Canemco Inc.). Samples were washed with 0.1 M sodium cacodylate buffer, pH 7.3, postfixed with 1% osmium tetroxide in 0.1 M sodium cacodylate buffer, pH 7.3, dehydrated in graded ethanols, and embedded in spurr epoxy resin (Canemco Inc.). Embedded tissues were sectioned with an RMC 6000 ultramicrotome, (RMC-EM, Tucson, Ariz.), stained with uranyl acetate, lead citrate, and examined in a Philips CM 100 electron microscope (Philips Electron Optics, Eindhoven, the Netherlands).

Fresh PRP was immediately fixed with 2% gultaraldehyde (Canemco Inc.) in 0.1 M sodium cacodylate buffer, pH 7.3 (Canemco Inc.) and then followed on the method described previously (Berger, at al., 1996).

For immunogold staining, freshly excised spleens obtained from 3- to 5-week-old Hzf^{-/-} and control mice were fixed in 0.1 M sodium cacodylate buffer, pH 7.3 containing a combination of 0.1% glutaraldehyde (Canemco Inc.) and 4% paraformaldehyde (Canemco Inc.). The samples were washed with the same buffer, dehydrated with graded ethanol, infiltrated in lowicryl (Canemco Inc.), and polymerized under UV at −20° C. over night. The sections were rinsed with PBS containing 0.15% glycine and 0.5% BSA, washed with 0.5% BSA in PBS, and then reacted with anti-PDGF-A antibody (Santa Cruz Biotechnology, Santa Cruz, Calif.) for 1 hr at 22° C. After washing with 0.5% BSA in PBS, the sections were treated with anti-rabbit IgG conjugated with 10 nm gold (Amersham, England). After washing with 0.5% BSA in PBS, PBS, and H₂O, the sections were stained with uranyl acetate, lead citrate, and examined in the Philips CM 100 electron microscope.

Western Blot Analysis

Bone marrow cells were isolated from 4-week-old Hzf^{+/+} and Hzf^{-/-} mice, after washed with PBS, subsequently suspended in lysis buffer (Nieswandt et al., 1999). Soluble fractions from each sample were boiled with sample buffer for SDS-PAGE. Eluted proteins were separated by electrophoresis and transferred to nitrocellulose membranes (Schleicher & Schuell, Keene, N. H.). Immunoblotting was performed as previously described (Nieswandt et al., 1999). AChE expression was evaluated to ensure equivalent sample loading. Commercially available antibodies to vWF (DAKO, Denmark), fibrinogen (ICN Biochemicals, Aurora, Ohio.), PDGF-A (Santa Cruz Biotechnology, Santa Cruz, Calif.), PDGF-B (Santa Cruz), and AChE (Santa Cruz) were used at the concentrations of 57 μg/ml, 160 μg/ml, 5 μg/ml, 5 μg/ml, and 5 μg/ml, respectively. Goat anti-rabbit IgG antibody conjugated with HRP and donkey anti-goat IgG antibodies conjugated with HRP were purchased from Bio-Rad (Hercules, Calif.) and Santa Cruz, respectively. The signals were visualized with an enhanced chemiluminescence detection system (Amersham, England) as directed by the manufacturer.

The detection of platelet proteins was carried out basically as described previously (Nieswandt et al., 1999). Briefly, washed platelets were lysed with the same lysis buffer, as described above. Whole platelets were treated with standard sample buffer and loaded onto 10% SDS-PAGE following the same protocol as described above. GPIIb was evaluated to ensure equivalent platelet protein loading. The anti-mouse GPIIb monoclonal antibody (mAb) and anti-rat IgG antibody conjugated with HRP were purchased from Pharmingen (La Jolla, Calif.) and Santa Cruz, respectively. The anti-GPIIb mAb and the anti-rat IgG antibody conjugated with HRP were used at the concentrations of 0.25 μg/ml and 0.8 μg/ml, respectively. The signal-enhancement was performed as described above.

RT-PCR Analysis

Bone marrow cells, from male Hzf^{+/+} and Hzf^{-/-} mice, were used for RNA extraction and cDNA synthesis, as described previously (Kimura et al., 1998). The detailed method for RT-PCR and the primer sequences were described elsewhere (Anderson et al., 1999; Fennie et al., 1995; Keller et al., 1993; Shivdasani et al., 1995; Vyas et al., 1999). cDNAs from male Hzf^{+/+} and Hzf^{-/-} bone marrow cells were equalized by RT-PCR of hypoxanthine phosphoribosyle transferase (HPRT). The RT-PCR products were analyzed by polyacrylamide gel electrophoresis.

Results

Generation of Hzf^{-/-} Mutant Mice

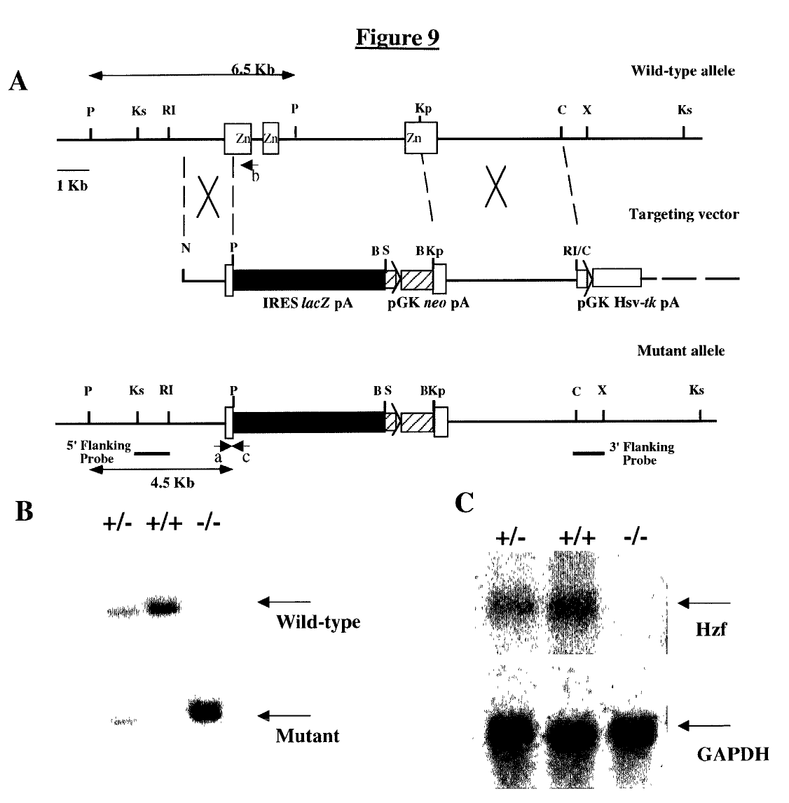

The Hzf gene was disrupted in murine ES cells using a targeting vector in which the exons encoding three zinc finger motifs were deleted (see above and FIG. 9A). The targeting vector was electroporated into R1 ES cells. Six independent clones were found to be heterozygous for the mutation at the Hzf locus. Three out of the 6 heterozygous mutant ES clones were used to generate chimeric mice. Chimeras were backcrossed to CD1 mice to generate mice heterozygous for the Hzf mutation. Heterozygous Hzf^{+/-} mice were fertile and were intercrossed to generate homozygous Hzf^{-/-} mice (FIG. 9B). The null mutation of Hzf was confirmed by the absence of Hzf expression, as determined by Northern blot analysis of RNA extracted from brains of Hzf mutant mice at 3 weeks after birth (FIG. 9C).

Hemorrhage and Neonatal Lethality of Hzf^{-/-} Mice

Initial examination of the progeny from Hzf^{+/-} parents revealed a reduced frequency of Hzf^{-/-} mice at 3 weeks of age. To determine the stage of neonatal development affected by the Hzf mutation, neonatal genotyping was performed. Of 300 offspring derived from heterozygous matings, 1 week-old Hzf^{-/-} pups were viable, usually of normal size, and were present in the expected Mendelian frequency. However, thereafter, especially between 2 to 3 weeks after birth, the percentage of homozygotes decreased (data not shown).

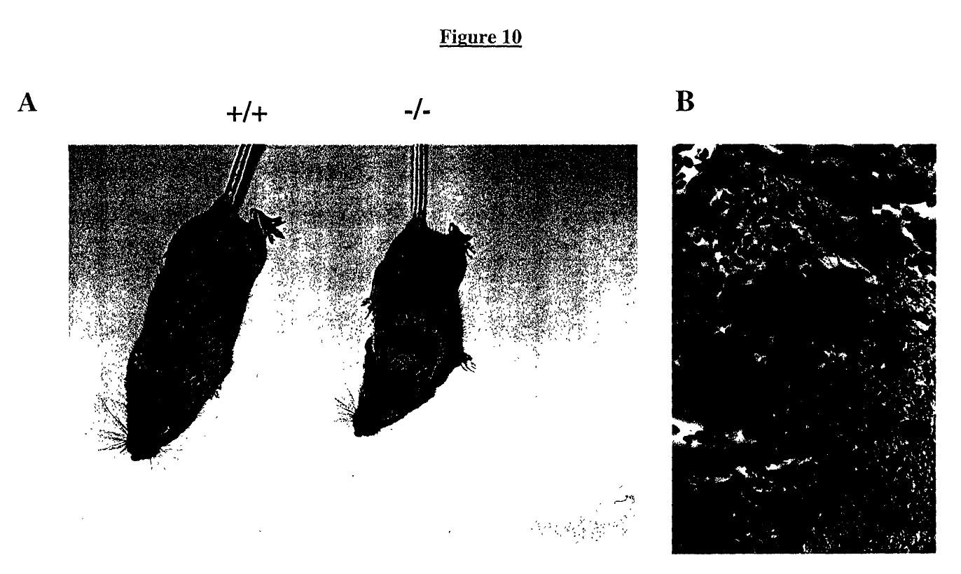

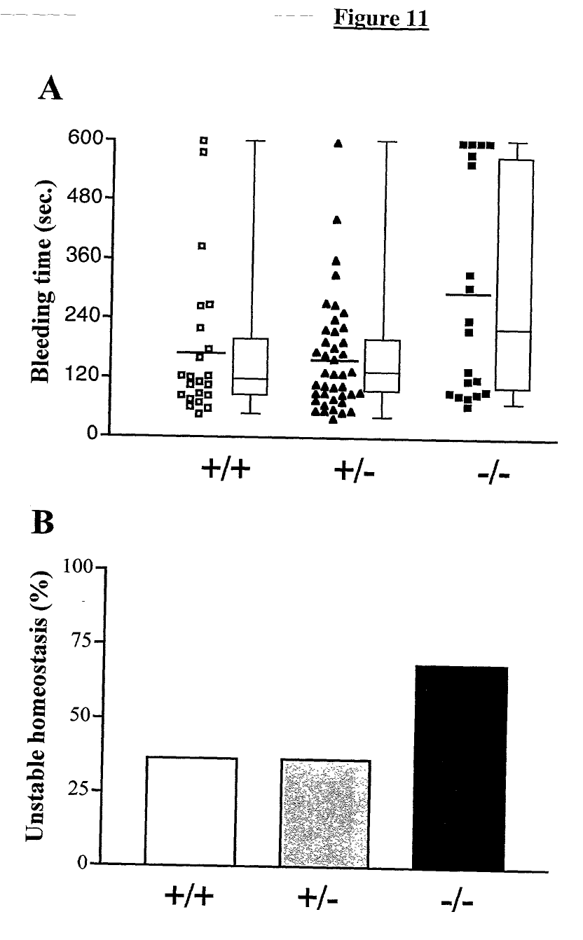

As shown in FIG. 10A, 4 week-old Hzf^{-/-} mice were distinguishable from their wild type littermates by sizes. Moreover, there was marked internal hemorrhaging evident primarily in the brains and gastrointestinal tracts (FIG. 10B and data not shown) in mutant neonates. This hemorrhaging was the likely cause of neonatal lethality. The surviving Hzf^{-/-} mice of either sex were fertile (data not shown).

Unstable Hemostatic Plug Formation and Abnormal Platelet Morphology in Hzf^{-/-} Mice

To determine whether the hemorrhaging phenotype was related to defective hemostasis in the Hzf-deficient mice, the bleeding times and rebleeding occurrences of Hzf^{+/+}, Hzf^{+/-}, and Hzf^{-/-} mice were compared. There was no significant difference in bleeding times when Hzf^{-/-} mice were compared with wild type and Hzf^{+/-} heterozygous mice (FIG. 11A). However, Hzf^{-/-} mice re-bled after transient hemostasis relative to the rebleeding frequency in wild type and Hzf^{+/-} mice (FIG. 11B). These results indicate that Hzf-deficiency leads to unstable hemostatic plug formation in vivo.

The defects in hemostasis in Hzf^{-/-} mice led to the analysis of hematologic parameters in Hzf^{-/-} mice. Complete hematologic profiles revealed no significant differences in platelet counts and differential counts between Hzf^{-/-} mice and their control littermates (data not shown).

To address the defect of hemostasis in detail, the morphology of blood cells in peripheral blood smears from Hzf^{-/-} mice was examined. Normal platelet sizes from control mice were observed by comparison with the diameter of erythrocytes (FIG. 11C). In contrast, platelets from Hzf^{-/-} mice often appeared large and were faint in color (FIG. 11D). Although morphologically normal platelets in Hzf^{-/-} peripheral blood were seen, the large faintly stained platelets, which were observed in FIG. 11D, were not detected in peripheral blood smears from Hzf^{+/+} mice. To examine these morphological differences more closely, ultrastructural analysis of peripheral platelets from Hzf^{-/-} and control mice was performed. As shown in FIG. 11E, wild type mice had normal dense α-granules, which could be easily identified, in platelets. In contrast, Hzf^{-/-} platelets had significantly reduced numbers of α-granules, with many vacuoles (FIG. 11F, panels a-d), suggesting that platelets in Hzf^{-/-} mice have defect of α-granule formation.

Numerous procoagulant substances are packed in platelet α-granules, and the release of the granule-contents is important for platelet function (Ware, 1995). vWF, one of the procoagulant substances contained in platelet α-granules, is associated with attachment of platelets onto damaged blood vessels, thereby reinforcing the stability of the plug, and activating coagulation pathway inducing blood clot (Ware, 1995). To investigate whether α-granules in Hzf^{-/-} platelets contain (C-granule substances, the protein levels of platelet-vWF, a key glycoprotein involved in coagulation, was examined. As shown in FIG. 11G, the levels of vWF were dramatically reduced in Hzf^{-/-} platelets when compared with that in platelets from wild type mice.

Number and DNA Ploidy Levels of Megakaryocytes and Megakaryocyte Progenitors from Hzf^{-/-} Mice

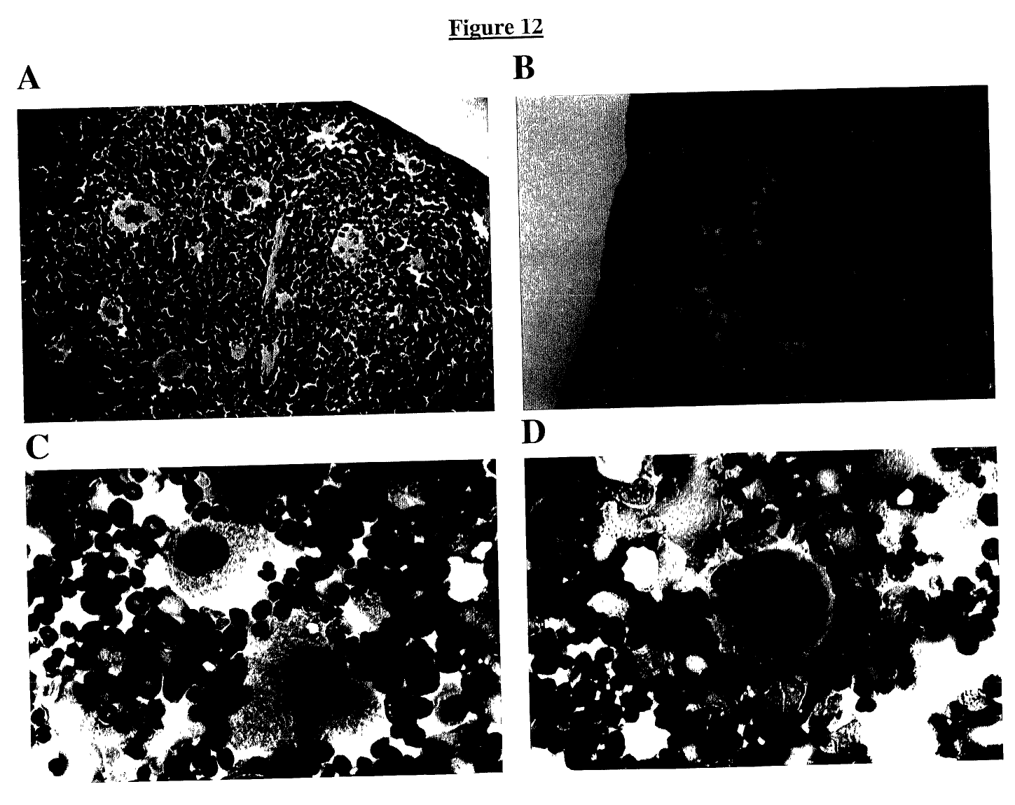

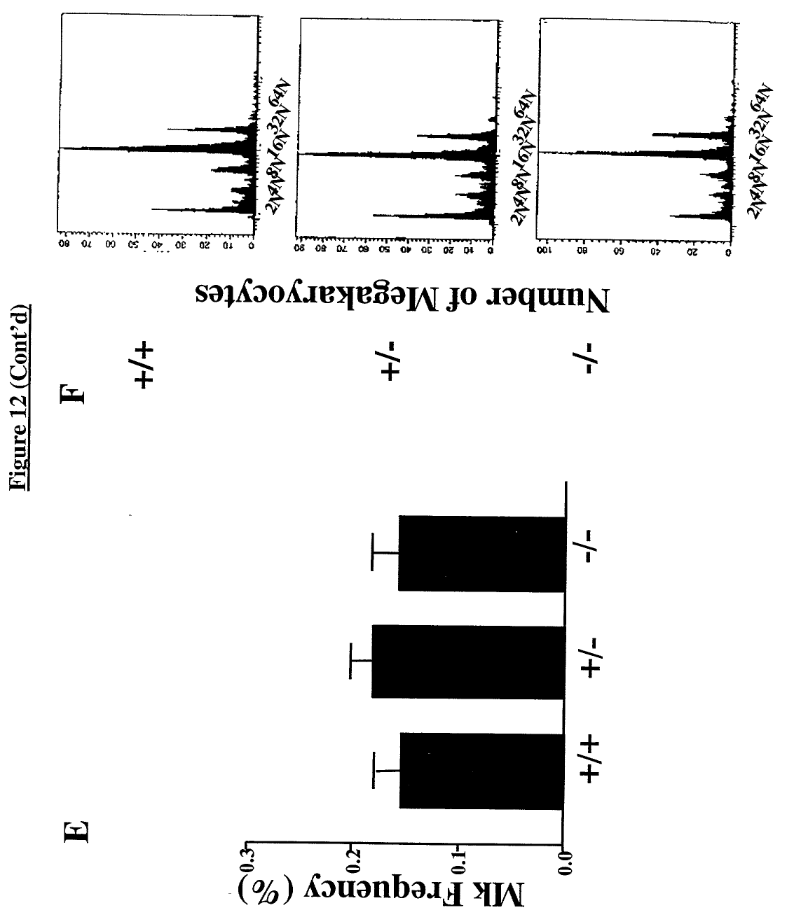

To explore in greater detail the nature of the platelet defects described above, megakaryocytes from Hzf^{-/-} mutant mice were characterized. Megakaryocytes populated, at the same frequency as that observed in control mice, in the spleens and bone marrows of Hzf mutant mice, as determined morphologically and by flow cytometric analysis (FIG. 12A-E).

Normal megakaryocyte development initiates with proliferation of precursor cells. These precursor cells then undergo endomitosis, followed by cytoplasmic maturation and organization, which culminates in platelet release (Zuker-Franklin, 1989). To determine whether the process of endomitosis was affected by the absence of Hzf, DNA ploidy analysis was performed on bone marrow megakaryocytes. As shown in FIG. 12F, no significant differences in DNA ploidy patterns were detected between wild type, heterozygous, and homozygous mutant mice. The levels of megakaryocyte progenitor cells (CFU-Mk) were examined in the bone marrow of Hzf wild type and mutant mice by plating bone marrow cells in semi-solid media in the presence of TPO/MGDF. Normal numbers of megakaryocyte progenitors were observed in Hzf-deficient mice (data not shown).

Ultrastructural Analysis of Megakaryocytic Abnormality

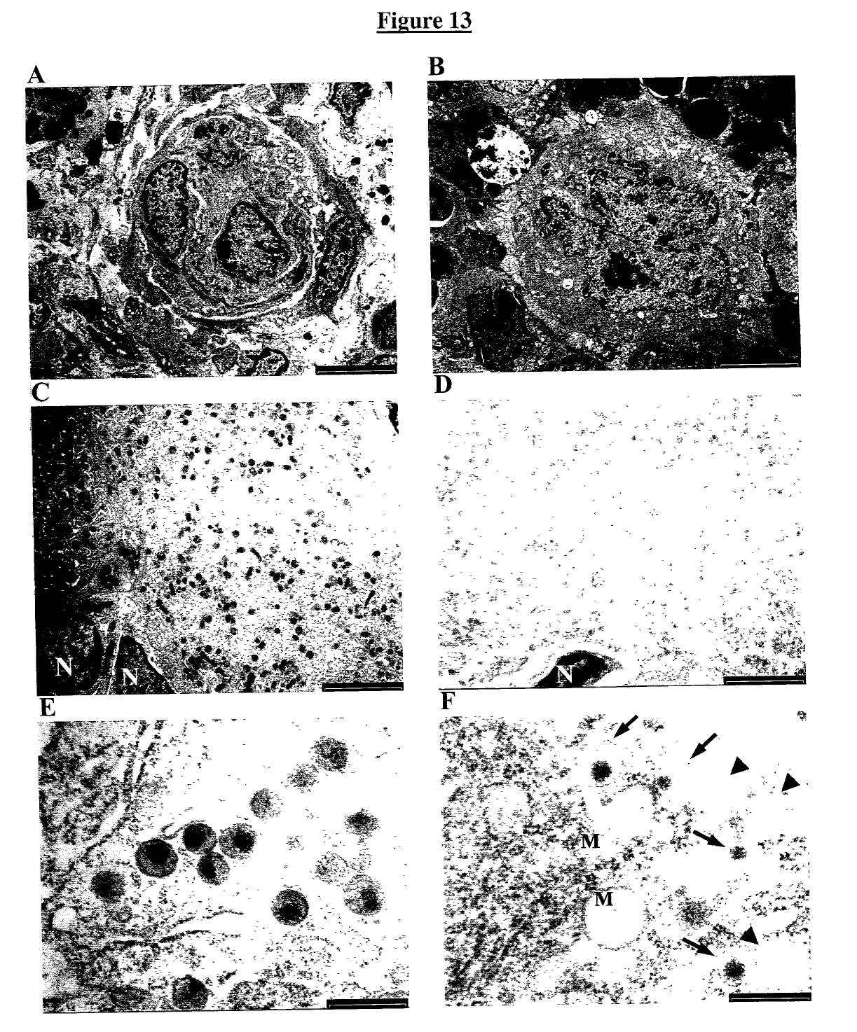

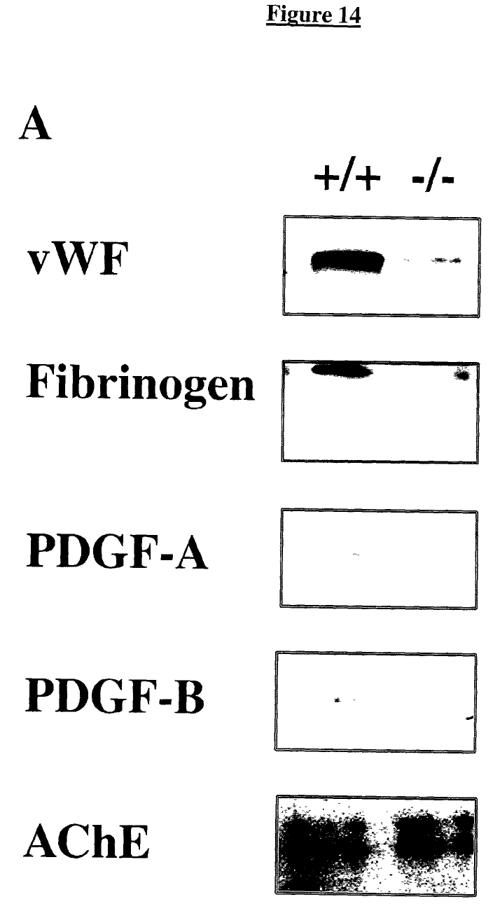

To characterize further the maturation defect in Hzf^{-/-} mice, electron microscopic analysis was performed on megakaryocytes from Hzf^{-/-} mice. As expected, megakaryocytes, from both Hzf^{+/+} and Hzf^{-/-} mice exhibited hyperlobulated nuclei (FIG. 13A and 13B). Megakaryocytes from Hzf^{+/+} mice exhibited well-formed platelet fields, demarcation membrane systems, and numerous α-granules (FIG. 13C); in contrast, Hzf^{-/-} megakaryocytes displayed a dramatic reduction in the number of α-granules, with many vacuoles, in their cytoplasm (FIG. 13D). Furthermore, detailed ultrastructural analysis revealed that α-granules in megakaryocytes from control mice were dense, readily identifiable, and exhibited distinct zones: dense nucleoid regions and diffuse granular matrixes (FIG. 13E). However, the inside of α-granules in Hzf^{-/-} megakaryocytes appeared to be pale and vacant although membranes of α-granules could be identified (FIG. 13F). In addition, the majority of Hzf^{-/-} α-granules was devoid of diffuse granular matrixes although dense nucleoid regions were observed only in some of α-granules in Hzf^{-/-} megakaryocytes (FIG. 13F). These observations suggest a defect in storage of α-granule substances in Hzf^{-/-} megakaryocytes.

Taken together, the data indicate that the process of endomitosis is not affected by Hzf-deficiency. However, Hzf-deficiency appears to block the process of terminal maturation of megakaryocytes, affecting the assembly of α-granule substances in α-granules of megakaryocytes.

Reduced Concentrations of α-Granule Substances in Hzf^{-/-} Megakaryocytes

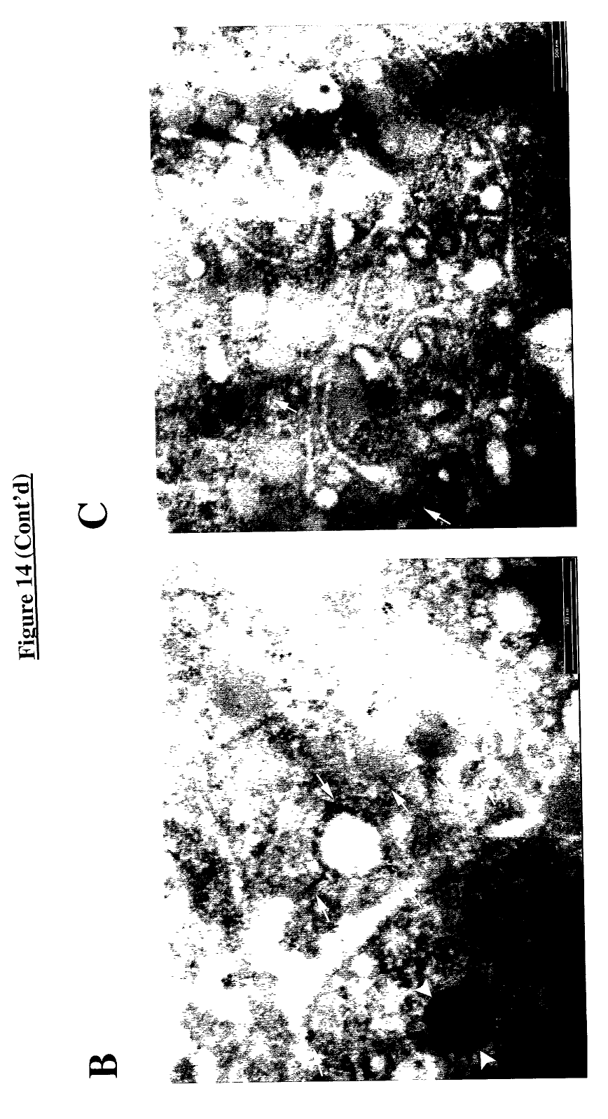

Previous biochemical and immunoelectron microscopic analyses have shown that megakaryocytic α-granules contain numerous substances essential to the coagulation system (Burstein, 1995). To examine the effect of Hzf-deficiency on production of α-granule substances in megakaryocytes, the levels of vWF, fibrinogen, PGDF-A, and PDGF-B were analyzed by Western immunoblotting. As shown in FIG. 14A, the levels of vWF and fibrinogen, which are adhesive glycoproteins, were both dramatically reduced in Hzf^{-/-} bone marrow. In addition, Hzf-deficient bone marrow showed significantly decreased protein levels of PDGF-A and PDGF-B, which are also α-granule substances.

To examine further the effect of Hzf-deficiency on α-granule substances in megakaryocytes, immunogold labeling of α-granule substances was performed followed by transmission electron microscopy. Using this strategy, concentrated gold particles recognizing PDGF-A were detected in α-granules and on vesicles in the cytoplasm of Hzf^{+/+} megakaryocytes (FIG. 14B). In contrast, PDGF-A was not detected in α-granules of Hzf^{-/-} megakaryocytes although a few gold particles were present on vesicles (FIG. 14C).

Down-Regulation of Megakaryocyte-Specific Genes

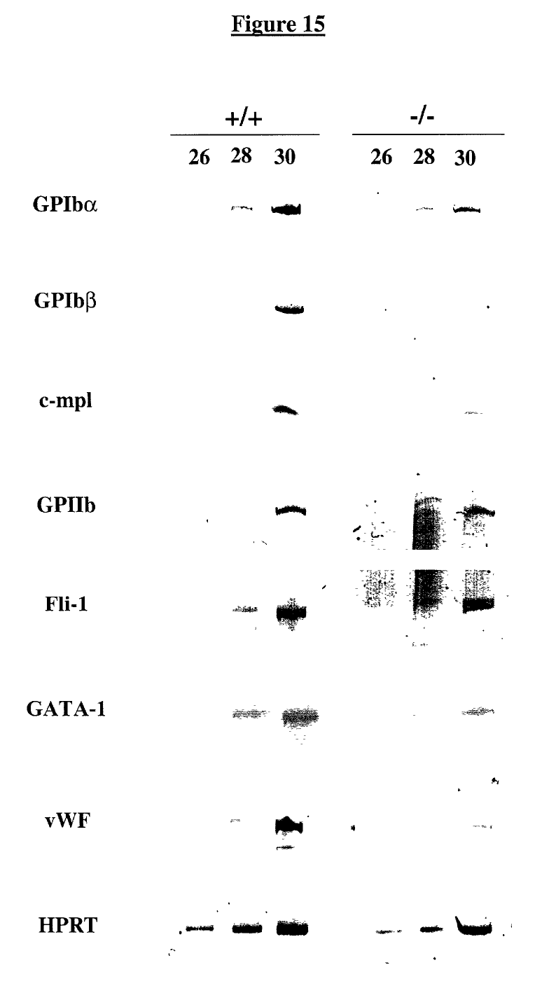

To assess whether the structural defects observed in megakaryocytes from Hzf^{-/-} mice were the consequence of an earlier block in megakaryocyte maturation, the expression of genes encoding transcription factors and cell surface receptors that are associated with megakaryocyte maturation were investigated (Hart et al., 2000; Vyas et al., 1999). In this experiment, bone marrow cells from male mutant and wild type mice were used as the source of mRNAs for comparison of expression levels of GATA-1 and Fli-1, two transcription factors known to be involved in megakaryopoiesis, vWF, a coadhesive glycoprotein, and GPIbα, GPIbβ, and c-mpl, and GPIIb, cell surface receptors expressed on megakaryocytes. As shown in FIG. 15, Hzf^{-/-} megakaryocytes expressed equivalent levels of GPIbα, GPIIb, GATA-1, and Fli-1 and relatively reduced levels of c-mpl as Hzf^{+/+} cells. In contrast, transcripts for vWF and GPIbβ genes in Hzf^{-/-} cells were markedly reduced relative to that observed in wild type cells.

Discussion

Hzf-deficiency causes a block in the formation of α-granules in megakaryocytes, abnormal platelet morphology, and disruption of hemostasis in vivo. These findings identify Hzf as a novel regulator of megakaryopoiesis and hemostasis.

Megakaryocyte Differentiation and α-Granule Formation in Hzf-Deficient Mice

Megakaryocytes in Hzf^{-/-} mice were produced in normal number and appeared to undergo complete maturation based on DNA ploidy analysis. Electronmicroscopic analysis revealed the presence of polyploid nuclei and the characteristic demarcation membrane system in megakaryocytes lacking HZF. Together, these findings suggest that Hzf-deficiency does not cause a block in the middle and late stages of megakaryocyte development. The numbers of megakaryocyte progenitor cells were also not affected by the absence of Hzf, suggesting no inhibition of the early stage of megakaryocyte development. Therefore, Hzf is not essential for megakaryocyte development.

The electronmicroscopic and biochemical analyses revealed that platelets and megakaryocytes from Hzf^{-/-} mice contain numerous vacuoles with the reduced amount of α-granule substances. Interestingly, membranes of α-granules were observed in Hzf^{-/-} megakaryocytes. In addition, PDGF-A was not detected in α-granules of Hzf^{-/-} megakaryocytes although a few gold particles were present on vesicles. These findings suggest that Hzf is required for the synthesis of α-granule substances and/or the assembly of the α-granule substances into α-granule bags.

Hzf in Platelet Morphogenesis and Hemostasis

Most Hzf^{-/-} mice were smaller than their littermates, presumably as a result of sustained hemorrhage during a period of rapid growth. Because the number of platelets in Hzf mutant was within normal range, HZF does not appear to impair the release of platelets from megakaryocytes. Nevertheless, circulating large, faintly stained platelets containing numerous empty vacuoles were present in the peripheral blood of Hzf^{-/-} mice. Mice carrying megakaryocyte-specific GATA- 1-deficiency exhibit reduced numbers of platelets and increase in size of circulating platelets (Shivdasani et al., 1997). GPIbα-deficient mice exhibit a bleeding disorder associated with circulating giant platelets (Ware et al., 2000). Hzf^{-/-} mice have normal levels of GATA-1 and GPIbα transcripts in bone marrow cells. Thus, these results suggest that the megakaryopoiesis defect in Hzf-deficient mice is independent of the roles of GATA-1 and GPIbα in platelet development function and that Hzf is not upstream of the genes that encode these proteins. Alternatively, it is possible that Hzf and GATA-1 act in concert and together are required for the expressions of GPIbα, vWF, and possibly other downstream genes.

Platelets from Hzf^{-/-} mice have dramatically reduced levels of the coadhesive glycoprotein, vWF. In addition, Hzf^{-/-} megakaryocytes had dramatically reduced levels of transcripts for GPIbβ. vWF and fibrinogen contribute to hemostasis by producing a platelet plug and then reinforcing the plug by converting fibrinogen to fibrin strands, inducing activation of coagulation pathway (Savage et al., 1996; Ware, 1995). It is also known that GPIb, which is a heterodimer of GPIbα and GPIbβ, has an important role in the adherence of platelets to subendothelial surfaces with binding to vWF (Parker and Gralnick, 1987; Sakariassen et al., 1986; Savage et al., 1996). Indeed, vWF^{-/-} platelets exhibit delayed adhesion to vessel walls and they failed to form thromba in arterioles in vivo (Denis et al., 1998; Ni et al., 2000). Thus, the instability of plug formation in Hzf-deficient mice may result from a failure of platelet adhesion onto damaged blood vessel walls, a process which is mediated and immobilized by vWF binding to GPIb.

Gene Regulation by Hzf

The generation of mice with targeted mutation in specific genes has greatly enhanced the understanding of megakaryocyte differentiation. Transcription factors including GATA-1 (Shivdasani et al., 1997; Vyas et al., 1999), FOG (Tsang et al., 1998), MafG (Shavit et al., 1998), and compound MafG::MafK (Onodera et al., 2000) are required for megakaryocyte differentiation. Fli-1 and GATA-1 may cooperate in regulating megakaryopoiesis, based on the observation that loss of function mutations in either gene results in reduced α-granules and loss of organization of platelet demarcation membranes (Hart et al., 2000). There are also several reports showing that megakaryocyte-specific gene expression may require both GATA-1 and a member of the Ets family of transcription factors (Lemarchandel et al., 1993; Romeo et al., 1990). For example, c-mpl, vwf, and gpIIb are directly regulated by Ets proteins in vitro (Mignotte et al., 1994; Schwachtgen et al., 1997; Zhang et al., 1993) and the promoters for the GPIbα, GPIbβ, and c-mpl genes include functional GATA-1 bind sites (Deveaux et al., 1996; Hashimoto and Ware, 1995; Yagi et al., 1994). Both GATA-1 and Fli-1 messages were expressed normally in bone marrow cells derived from Hzf^{-/-} mice, suggesting that Hzf may not affect megakaryocyte differentiation through GATA-1 and/or Fli-1. In contrast, RNA transcripts for the vWF and GPIbβ genes were significantly reduced in bone marrow cells from Hzf^{-/-} mice. Thus, these results suggest that Hzf may regulate the expression of vwf and gpIbβ independently of both GATA- 1 and Fli-1.

Taken together, these data indicate that Hzf plays an essential role in hemostasis in vivo through its effects on the levels of at least two molecules, vWF and GPIbβ. The defect in hemostasis is associated with dramatically reduced levels of vWF and GPIbβ, two proteins that are known to be involved in plug formation through the adhesion of platelets to endothelial cells. The defects in Hzf-deficient mice reflect, at least in part, a role for Hzf in the regulation of the levels of these two proteins. The levels of another transcription factor, GATA-1, known to be involved in megakaryopoiesis, was unaffected in Hzf-deficient mice, suggesting that Hzf is not upstream of GATA-1, but rather that Hzf alone, or in concert with GATA-1, regulates expression of the vwf and gpIbβ genes.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

TABLE 1
Summary of lacZ expression within ES cell gene trap clones
upon hema-topoietic differentiation^{a}

| | Days after induction | | | |
|---|---|---|---|---|
| Number of clones | 0 | 5 | 10 | |
| White clones | | | | |
| 1278 | − | − | − | |
| 1 | − | + | − | 7H7 |
| 1 | − | + | + | Hh1 |
| Blue clones | | | | |
| 2 | + | + | + | 4A11 and Hzf |
| 24 | + | + | − | |
| 44 | + | − | − | |
| 1350 total | | | | |

^{a}White clones, lacZ negative in undifferentiated ES cells; blue clones, lacZ positive in undifferentiated ES cells; +, lacZ positive during differentiation; −, lacZ negative during differentiation.

Full Citations of References Cited in the Specification

1. Bernstein A, Forrester L, Reith A D, Dubreuil P, Rottapel R: The murine W/c-kit and Steel loci and the control of hematopoiesis. Semin. Hematol. 28:138-142, 1991

2. Bedell M A, Jenkins N A, Copeland N G: Mouse models of human disease. Part I: Techniques and resources for genetic analysis of mice. Genes & Dev. 11:1-10, 1997

3. Bedell M A, Largaespada D A, Jenkins N A, Copeland N G: Mouse models of human disease. Part II: Recent progress and future directions. Genes & Dev. 11: 11-43, 1997

4. Shivdasani R A and Orkin S H: The transcriptional control of hematopoiesis. Blood 87: 4025, 1996

5. Bernstein A, Correll P, Paulson R, Potter M: In vitro and in vivo approaches to the molecular analysis of mouse hematopoiesis. In Mammalian Development ed Peter Lonai, pg 101, 1996

6. Fischer, K-D Zmuidzinas A, Gardner S, Barbacid M, Bernstein A Guidos C. Defective T-cell receptor signalling and positive selection of vav-deficient CD4+CD8+thymocytes. Nature 374:474, 1995

7. Shalaby F, Rossant J, Yamaguchi T P, Gertstenstein M, Wu X-F, Breitman M L, Shuh A C: Failure of blood-island formation and vasculogenesis in Flk-1-deficient mice. Nature 376:62, 1995

8. Gurney A L., Carver-Moore K, de Sauvage F J, Moore M W: Thrombocytopenia in c-mpl deficient mice. Science 265:1445, 1994

9. Cheng J, Baumhueter S, Cacalano, G Thibodeaux H, Thomas R, Broxmeyer H E, Cooper S, Hague N, Moore M, Lasky L A: Hematopoietic defects in mice lacking the sialomucin CD34. Blood 87:479, 1996.

10. Lieschke G J, Grail D, Hodgson G, Metcalf D, Stanley E, Cheers C, Fowler K J, Basu S, Zhan Y K, Dunn A R: Mice lacking granulocyte colony-stimulating factor have chronic neutropenia, granulocyte and macrophage progenitor cell deficiency, and impaired neutrophil mobilization. Blood 84: 1737, 1994

11. Gossler A, Joyner A L, Rossant J, Skarnes W C: Mouse embryonic stem cells and reporter constructs to detect developmentally regulated genes. Science 244: 463, 1989

12. Friedrich G and Soriano P: Promoter traps in embryonic stem cells: A genetic screen to identify and mutate developmental genes in mice. Genes & Dev, 5: 1513, 1991

13. Skarnes W C, Auerbach B A, Joyner A L: A gene trap approach in mouse embryonic stem cells: the lacZ reporter is activated by splicing, reflects endogenous gene expression, and is mutagenic in mice. Genes & Dev. 6: 903, 1992

14. von Melchner H, DeGregori J V, Rayburn H, Reddy S, Friedel C, Ruley H E. Selective disruption of genes expressed in totipotent embryonal stem cells. Genes & Dev. 6: 919, 1992

15. Wurst W, Rossant J, Prideaux V, Kowanacka M, Joyner A L, Hill D P, Guillemot F, Gasca S, Cado D, Auerbach A, Ang S-L. A large-scale gene-trap screen for insertional mutations in developmentally regulated genes in mice. Genetics 139:889, 1995

16. Evans M J, Carlton M B L, Russ A P. Gene trapping and functional genomics. Trends Genet. 13:370, 1997

17. Sam M, Wurst W, Klüppel M, Jin O, Heng H, Bernstein A: Aquarius, a novel gene isolated by gene trapping with an RNA-dependent RNA polymerase motif. Dev. Dyn. 212: 304, 1998

18. Stanford W L, Caruana G, Vallis K A, Inamdar M, Hidaka M, Bautch V L, Bernstein A: Expression trappping: Identification of novel genes expressed in hematopoietic and endothelial lineages by gene trapping in ES cells. Blood 92: 4622, 1998.

19. Doetschman T C, Eistetter H, Katz M, Schmidt W, Kemler R: The in vitro development of blastocyst-derived embryonic stem cell lines: Formation of visceral yolk sac, blood islands and myocardium. J. Embryol. Exp. Morphol. 87: 27, 1985

20. Bain G, Kitchens D, Yao M, Huettner J E, Gottlieb D I: Embryonic stem cells express neuronal properties in vitro. Dev. Biol.168:342, 1995

21. Maltsev V A, Rohwedel J, Hescheler J, Wobus A M: Embryonic stem cells differentiate in vitro into cardiomyocytes representing sinusnodal, atrial and ventricular cell types. Mech. Dev. 44: 41, 1993

22. Wang R, Clark R, Bautch V L: Embryonic stem cell-derived cystic embryoid bodies form vascular channels: an in vitro model of blood vessel development. Development 114:303, 1992

23. Rohwedel J, Maltsev V, Bober E, Arnold H-H, Hescheler J, Wobus A M: Muscle cell differentiation of embryonic stem cells reflects myogenesis in vivo: Developmentally regulated expression of myogenic determination genes and functional expression of ionic currents. Dev. Biol. 164:87, 1994.

24. Keller G, Kennedy M, Papayannopoulou T, Wiles M V: Hematopoietic commitment during embryonic stem cell differentiation in culture. Mol. Cell. Biol. 13: 473, 1993

25. Wiles M V and Keller G: Multiple hematopoietic lineages develop from embryonic stem (ES) cells in culture. Development 111: 259, 1991

26. Schmitt RM, Bruyns E, Snodgrass R: Hematopoietic development of embryonic stem cells in vitro: cytokine and receptor gene expression. Genes & Development 5: 728, 1991

27. Palacios R, Bucana C, Xie X: Long-term culture of lymphohematopoietic stem cells. Proc. Natl. Acad. Sci. USA 93: 5247, 1996

28. Kennedy M, Firpo M, Choi K, Wall C, Robertson S, Kabrun N, Keller G: A common precursor for primitive erythropoiesis and definitive haematopoiesis. Nature 386: 488, 1997

29. Forrester L M, Nagy A, Sam M, Watt A, Stevenson L, Bernstein A, Joyner A L, Wurst W: An induction gene trap screen in embryoid stem cells: Identification of genes that respond to retinoic acid in vitro. Proc. Natl. Acad. Sci. USA 93:1677, 1996

30. Shirai M, Miyashita A, Ishii N, Itoh Y, Satokata I, Watanabe YG, Kuwano R: A gene trap strategy for identifying genes expressed in the embryonic nervous system. Zool. Sci. 13:277, 1996

b −Baker R K, Haendel M A, Swanson B J, Shambaugh J C, Micales B K, Lyons G E: In vitro preselection of gene-trapped embryonic stem cell clones for characterizing novel developmentally regulated genes in the mouse. Dev. Biol. 185:201, 1997

32. Muth K, Bruyns R, Thorey I S, von Melchner H: Disruption of genes regulated during hematopoietic differentiation of mouse embryonic stem cells. Dev Dyn. 212: 277, 1998

33. Nakano T, Kodama H, Honjo T: Generation of lymphohematopoietic cells from embryonic stem cells in culture. Science 265: 1098, 1994

34. Nagy A R J, Rossant J, Nagy R, Abramow-Newerly W, Roder J C: Derivation of completely cell culture-derived mice from early passage embryonic stem cells. Proc. Natl. Acad. Sci. USA. 90: 8424, 1993

35. Nagy A R J: In Gene Targeting: A Practical Approach, J.A.L., ed (Oxford: IRL), pp. 147-178 (1993)

36. Hill D and Wurst W: Screening for novel pattern formation genes using gene trap approaches. Methods Enzymol. 225: 664, 1993

37. von Melchner H, Reddy S, Ruley H E. Isolation of cellular promoters by using a retrovirus promoter trap. Proc Natl Acad Sci U S A. 87: 3733, 1990

38. Sambrook J, Fritsch E F, Maniatis T: Molecular Cloning. A Laboratory Manual. Cold Spring Harbor: Cold Spring Harbor Laboratory Press, 1989

39. Fiering S N, Roederer M, Nolan G P, Micklem D R, Parks D R, Herzenberg L A: Improved FACS-Gal: Flow cytometric analysis and sorting of viable eukaryotic cells expressing reporter gene constructs. Cytometry 12:291, 1991

40. Hogge D, Fanning S, Bockhold K, Petzer A, Lambie K, Lansdorp P, Eaves A, Eaves C: Quantitation and characterization of human megakaryocyte colony-forming cells using a standardized serum-free agarose assay. Brit. J. Haem. 96: 790, 1997.

41. Finerty P J and Bass B L: A Xenopus zinc finger polypeptide that specifically binds dsRNA and RNA-DNA hybrids. J. Mol. Biol. 271: 195, 1997

42. Varmeh-Ziaie S, Okan I, Wang Y, Magnusson K P, Warthoe P, Strauss M, Wiman K G: Wig-1, a new p53-induced gene encoding a zinc finger polypeptide. Oncogene 15: 2699, 1997

43. Israeli D, Tessler E, Haupt Y, Elkeles A, Wilder S, Amson R, Telerman A, Oren M: A novel p53-inducible gene, PAG608, encodes a nuclear zinc finger polypeptide whose overexpression promotes apoptosis. EMBO. 16:4384, 1997

44. Gasca S, Hill D P, Klingensmith J, Rossant J: Characterization of a gene trap insertion into a novel gene, cordon-bleu, expressed in axial structures of the gastrulating mouse embryo. Dev. Genetics 17: 141, 1995

45. Serafini T, Colamarino S A, Leonardo E D, Wang H, Beddington R, Skarnes W C, Tessier-Lavigne M: Netrin-1 is required for commissural axon guidance in the developing vetebrate nervous system. Cell 87: 1001, 199

46. Yeo T T, Yang T, Massa S M, Zhang J S, Honkaniemi J, Butcher L L, Longo F M: Deficient LAR expression decreases basal forebrain cholinergic neuronal size and hippocampal cholinergic innervation. J. Neurosci. Res. 47: 348, 1997

47. Faisst, A M and Gruss P: Bodenin: A novel murine gene expressed in restricted areas of the brain. Dev. Dyn. 212; 293, 1998

48. Voss A K, Thomas T, Gruss P: Compensation for a gene trap mutation in the murine microtubule-associated polypeptide 4 locus by alternative polyadenylation and alternative splicing. Dev Dyn. 212: 258, 1998

49. McClive P, Pall G, Newton K, Lee M, Mullins J, Forrester L: Gene trap integrations expressed in the developing heart: Insertion site affects splicing of the PT1-ATG vector. Dev Dyn. 212: 267, 1998

50. Melet F, Motro B, Rossi D J, Zhang L, Bernstein A: Generation of a novel Fli-1 polypeptide by gene targeting leads to a defect in thymus development and a delay in Friend virus-induced erythroleukemia. Mol. Cell. Biol. 16: 2708, 1996

51. Dzierzak E, and Medvinsky A: Mouse embryonic hematopoiesis. Trends Genet. 11: 359, 1995

52. Medvinsky A and Dzierzak E: Definitive hematopoiesis is autonomously initiated by the AGM region. Cell. 86: 897, 1996

53. deSauvage FJ, Carver-Moore K, Luoh S-M, Ryan A, Dowd M, Eaton D L, Moore M W. Physiological regulation of early and late stages of megakaryocytopoiesis by thrombopoietin. J. Exp Med 183:651, 1996

54. Alexander W S, Roberts A R, Nicola N A, Li R, Metcalf D: Deficiencies in progenitor cells of multiple hematopoietic lineages and defective megakaryocytopoiesis in mice lacking the thrombopoietin receptor c-Mpl. Blood 87:2162, 1996

55. Carver-Moore K, Broxmeyer H E, Luoh S-M, Ryan A, Dowd M, Eaton D L, Moore M W, deSauvage F J: Low levels of erythroid and myeloid progenitors in thrombopoietin and c-mpl-deficient mice. Blood 88:803, 1996

56. Shivdasani R A, Fukiwara Y, McDevitt M A, Orkin S H: A lineage-selective knockout establishes the critical role of transcription factor GATA-1 in megakaryocyte growth and platelet development. EMBO 16: 3965, 1997

57. Shivdasani R A, Rosenblatt M F, Zucker-Franklin D, Jackson C W, Hunt P, Saris C J, Orkin S H: Transcription factor NF-E2 is required for platelet formation independent of the actions of thrombopoietin/MGDF in megakaryocyte development. Cell 81:695, 1995

58. Fujiwara Y, Browne C P, Cunniff K, Goff S C, Orkin S H: Arrested development of embryonic red cell precursors in mouse embryos lacking transcription factor GATA-1. PNAS 93: 12355, 1996

59. Tsang A P, Visvader J E, Turner C A, Fujiwara Y, Yu C, Weiss M J, Crossley M, Orkin S H: FOG, a multiple zinc finger polypeptide, acts as a cofactor for transcription factor GATA-1 in erythroid and megakaryocytic differentiation. Cell 90: 109, 1997

60. Tsang A P, Fujiwara Y, Hom D B, Orkin S H: Failure of megakaryopoiesis and arrested erythropoiesis in mice lacking the GATA-1 transcriptional cofactor FOG. Genes and Dev 12:1176, 1998

61. Alexander, W. S., Roberts, A. W., Nicola, N. A., Li, R., and Metcalf, D. (1996). Deficiencies in progenitor cells of multiple hematopoietic lineages and defective megakaryocytopoiesis in mice lacking the thrombopoietic receptor c-Mpl. Blood 87, 2162-2170.

62. Anderson, M. K., Hernandez-Hoyos, G., Diamond, R. A., and Rothenberg, E. V. (1999). Precise developmental regulation of Ets family transcription factors during specification and commitment to the T cell lineage. Development 126, 3131-3148.

63. Arnold, J. T., Daw, N. C., Stenberg, P. E., Jayawardene, D., Srivastava, D. K., and Jackson, C. W. (1997). A single injection of pegylated murine megakaryocyte growth and development factor (MGDF) into mice is sufficient to produce a profound stimulation of megakaryocyte frequency, size, and ploidization. Blood 89, 823-833.

64. Bartley, T. D., Bogenberger, J., Hunt, P., Li, Y. S., Lu, H. S., Martin, F., Chang, M. S., Samal, B., Nichol, J. L., Swift, S., et al. (1994). Identification and cloning of a megakaryocyte growth and development factor that is a ligand for the cytokine receptor Mpl. Cell 77, 1117-1124.

65. Blank, V., and Andrews, N. C. (1997). The Maf transcription factors: Regulators of differentiation. Trends Biochem Sci 22, 437-441.

66. Burstein, S. A. and Breton-Gorius, J (1995). Megakaryopoiesis and platelet formation. In Williams Hematology, E. Beutler, Litchtman, M. A., Coller, B. S., and Kipps, T. J., ed. (USA, McGraw-Hill, Inc.), pp. 1149-1160.

67. Choi, E. S., Hokom, M. M., Chen, J. L., Skrine, J., Faust, J., Nichol, J., and Hunt, P. (1996). The role of megakaryocyte growth and development factor in terminal stages of thrombopoiesis. Br J Haematol 95, 227-233.

68. de Sauvage, F. J., Carver-Moore, K., Luoh, S.-M., Ryan, A., Dowd, M., Eaton, D. L., and Moore, M. W. (1996). Physiological regulation of early and late stages of megakaryocytopoiesis by thrombopoietin. J Exp Med 183, 651-656.

69. de Sauvage, F. J., Hass, P. E., Spencer, S. D., Malloy, B. E., Gurney, A. L., Spencer, S. A., Darbonne, W. C., Henzel, W. J., Wong, S. C., Kuang, W.-J., et al. (1994). Stimulation of megakaryocytopoiesis and thrombopoiesis by the c-Mpl ligand. Nature 369, 533-538.

70. Dejana, E., Quintana, A., Callioni, A., and de Gaetano, G. (1979). Bleeding time in laboratory animals. III—Do tail bleeding times in rats only measure a platelet defect? (the aspirin puzzle). Thromb Res 15, 199-207.

71. Denis, C., Methia, N., Frenette, P. S., Rayburn, H., Ullman-Cullere, M., Hynes, R. O., and Wagner, D. D. (1998). A mouse model of severe von Willebrand disease: Defects in hemostasis and thrombosis. Proc Natl Acad Sci U S A 95, 9524-9529.

72. Deveaux, S., Filipe, A., Lemarchandel, V., Ghysdael, J., Romeo, P.-H., and Mignotte, V. (1996). Analysis of the thrombopoietin receptor (MPL) promoter implicates GATA and Ets proteins in the coregulation of megakaryocyte-specific genes. Blood 87, 4678-4685.

73. Fennie, C., Cheng, J., Dowbenko, D., Young, P., and Lasky, L. A. (1995). CD34⁺ endothelial cell lines derived from murine yolk sac induce the proliferation and differentiation of yolk sac CD34⁺ hematopoietic progenitors. Blood 86, 4454-4467.

74. Greenberg, S. M., Kuter, D. J., and Rosenberg, R. D. (1987). In vitro stimulation of megakaryocyte maturation by megakaryocyte stimulatory factor. J Biol Chem 262, 3269-3277.

75. Gurney, A. L., Carver-Moore, K., de Sauvage, F. J., and Moore, M. W. (1994). Thrombocytopenia in c-mpl-deficient mice. Science 265, 1445-1447.

76. Hart, A., Melet, F., Grossfeld, P., Chien, K., Jones, C., Tunnacliffe, A., Favier, R., and Bernstein, A. (2000). Fli-1 is required for murine vascular and megakaryocytic development and is hemizygously deleted in patients with thrombocytopenia. Immunity 13, 167-177.

77. Hashimoto, Y., and Ware, J. (1995). Identification of essential GATA and Ets binding motifs within the promoter of the platelet glycoprotein Iba gene. J Biol Chem 270, 24532-24539.

78. Hidaka, M., Caruana, G., Stanford, W. L., Sam, M., Correll, P. H., and Bernstein, A. (2000). Gene trapping of two novel genes, Hzf and Hhl, expressed in hematopoietic cells. Mech Dev 90, 3-15.

79. Jackson, C. W., Brown, L. K., Somerville, B. C., Lyles, S. A., and Look, A. T. (1984). Two-color flow cytometric measurement of DNA distributions of rat megakaryocytes in unfixed, unfractionated marrow cell suspensions. Blood 63, 768-778.

80. Kato, T., Ogami, K., Shimada, Y., Iwamatsu, A., Sohma, Y., Akahori, H., Horie, K., Kokubo, A., Kudo, Y., and Maeda, E. (1995). Purification and characterization of thrombopoietin. J Biochem (Tokyo) 118, 229-236.

81. Kaushansky, K., Lok, S., Holly, R. D., Broudy, V. C., Lin, N., Bailey, M. C., Forstrom, J. W., Buddle, M. M., Oort, P. J., Hagen, F. S., et al. (1994). Promotion of megakaryocyte progenitor expansion and differentiation by the c-Mpl ligand thrombopoietin. Nature 369, 568-571.

82. Keller, G., Kennedy, M., Papayannopoulou, T., and Wiles, M. V. (1993). Hematopoietic commitment during embryonic stem cell differentiation in culture. Mol Cell Biol 13, 473-486.

83. Kimura, Y., Yamada, K., Sakai, T., Mishima, K., Nishimura, H., Matsumoto, Y., Singh, M., and Yoshikai, Y. (1998). The regulatory role of heat shock protein 70-reactive CD4⁺ T cells during rat listeriosis. Int Immunol 10, 117-130.

84. Kuter, D. J., Beeler, D. L., and Rosenberg, R. D. (1994). The purification of megapoietin: A physiological regulator of megakaryocyte growth and platelet production. Proc Natl Acad Sci U S A 91, 11104-11108.

85. Law, D. A., DeGuzman, F. R., Heiser, P., Ministri-Madrid, K., Killeen, N., and Phillips, D. R. (1999). Integrin cytoplasmic tyrosine motif is required for outside-in AIIbb3 signalling and platelet function. Nature 401, 808-811.

86. Lemarchandel, V., Ghysdael, J., Mignotte, V., Rahuel, C., and Romeo, P.-H. (1993). GATA and Ets cis-acting sequences mediate megakaryocyte-specific expression. Mol Cell Biol 13, 668-676.

87. Lok, S., Kaushansky, K., Holly, R. D., Kuijper, J. L., Lofton-Day, C. E., Oort, P. J., Grant, F. J., Heipel, M. D., Burkhead, S. K., Kramer, J. M., et al. (1994). Cloning and expression of murine thrombopoietin cDNA and stimulation of platelet production in vivo. Nature 369, 565-568.

88. Mignotte, V., Vigon, I., Boucher de Crevecoeur, E., Romeo, P. H., Lemarchandel, V., and Chretien, S. (1994). Structure and transcription of the human c-mpl gene (MPL). Genomics 20, 5-12.

89. Motohashi, H., Shavit, J. A., Igarashi, K., Yamamoto, M., and Engel, J. D. (1997). The world according to Maf. Nucleic Acids Res 25, 2953-2959.

90. Ni, H., Denis, C. V., Subbarao, S., Degen, J. L., Sato, T. N., Hynes, R. O., and Wagner, D. D. (2000). Persistence of platelet thrombus formation in arterioles of mice lacking both von Willebrand factor and fibrinogen. J Clin Invest 106, 385-392.

91. Nieswandt, B., Echtenacher, B., Wachs, F.-P., Schroder, J., Gessner, J. E., Schmidt, R. E., Grau, G. E., and Mannel, D. N. (1999). Acute systemic reaction and lung alterations induced by an antiplatelet integrin gpIIb/IIIa antibody in mice. Blood 94, 684-693.

92. Nye, J. A., Petersen, J. M., Gunther, C. V., Jonsen, M. D., and Graves, B. J. (1992). Interaction of murine ets-1 with GGA-binding sites establishes the ETS domain as a new DNA-binding motif. Genes Dev 6, 975-990.

93. Onodera, K., Shavit, J. A., Motohashi, H., Yamamoto, M., and Engel, J. D. (2000). Perinatal synthetic lethality and hematopoietic defects in compound mafG::mafK mutant mice. EMBO J 19, 1335-1345.

94. Parker, R. I., and Gralnick, H. R. (1987). Fibrin monomer induces binding of endogenous platelet von Willebrand factor to the glycocalicin portion of platelet glycoprotein lb. Blood 70, 1589-1594.

95. Romeo, P.-H., Prandini, M.-H., Joulin, V., Mignotte, V., Prenant, M., Vainchenker, W., Marguerie, G., and Uzan, G. (1990). Megakaryocytic and erythrocytic lineages share specific transcription factors. Nature 344, 447-449.

96. Sakariassen, K. S., Nievelstein, P. F. E. M., Coller, B. S., and Sixma, J. J. (1986). The role of platelet membrane glycoproteins Ib and Ib-Ia in platelet adherence to human artery subendothelium. Br J Haematol 63, 681-691.

97. Savage, B., Saldivar, E., and Ruggeri, Z. M. (1996). Initiation of platelet adhesion by arrest onto fibrinogen or translocation on von Willebrand factor. Cell 84, 289-297.

98. Schwachtgen, J. L., Janel, N., Barek, L., Duterque-Coquillaud, M., Ghysdael, J., Meyer, D., and Kerbiriou-Nabias, D. (1997). Ets transcription factors bind and transactivate the core promoter of the von Willebrand factor gene. Oncogene 15, 3091-3102.

99. Shavit, J. A., Motohashi, H., Onodera, K., Akasaka, J., Yamamoto, M., and Engel, J. D. (1998). Impaired megakaryopoiesis and behavioral defects in mafG-null mutant mice. Genes Dev 12, 2164-2174.

100. Shivdasani, R. A., Fujiwara, Y., McDevitt, M. A., and Orkin, S. H. (1997). A lineage-selective knockout establishes the critical role of transcription factor GATA-1 in megakaryocyte growth and platelet development. EMBO J 16, 3965-3973.

101. Shivdasani, R. A., Rosenblatt, M. F., Zucker-Franklin, D., Jackson, C. W., Hunt, P., Saris, C. J. M., and Orkin, S. H. (1995). Transcription factor NF-E2 is required for platelet formation independent of the actions of thrombopoietin/MGDF in megakaryocyte development. Cell 81, 695-704.

102. Souyri, M., Vigon, I., Penciolelli, J.-F., Heard, J.-M., Tambourin, P., and Wendling, F. (1990). A putative truncated cytokine receptor gene transduced by the myeloproliferative leukemia virus immortalizes hematopoietic progenitors. Cell 63, 1137-1147.

103. Stanford, W. L., Caruana, G., Vallis, K. A., Inamdar, M., Hidaka, M., Bautch, V. L., and Bernstein, A. (1998). Expression trapping: identification of novel genes expressed in hematopoietic and endothelial lineages by gene trapping in ES cells. Blood 92, 4622-4631.

104. Tsang, A. P., Fujiwara, Y., Hom, D. B., and Orkin, S. H. (1998). Failure of megakaryopoiesis and arrested erythropoiesis in mice lacking the GATA-1 transcriptional cofactor FOG. Genes Dev 12, 1176-1188.

105. Vyas, P., Ault, K., Jackson, C. W., Orkin, S. H., and Shivdasani, R. A. (1999). Consequences of GATA-1 deficiency in megakaryocytes and platelets. Blood 93, 2867-2875.

106. Ware, J., Russell, S., and Ruggeri, Z. M. (2000). Generation and rescue of a murine model of platelet dysfunction: The Bernard-Soulier syndrome. Proc Natl Acad Sci U S A 97, 2803-2808.

107. Ware, J. A. and Coller, B. S. (1995). Platelet morphology, biochemistry, and function. In Williams Hematology, E. Beutler, Litchtman, M. A., Coller, B. S., and Kipps, T. J., ed. (USA, McGraw-Hill, Inc.), pp. 1161-1201.

108. Wendling, F., Maraskovsky, E., Debili, N., Florindo, C., Teepe, M., Titeux, M., Methia, N., Breton-Gorius, J., Cosman, D., and Vainchenker, W. (1994). c-Mpl ligand is a humoral regulator of megakaryocytopoiesis. Nature 369, 571-574.

109. Yagi, M., Edelhoff, S., Disteche, C. M., and Roth, G. J. (1994). Structural characterization and chromosomal location of the gene encoding human platelet glycoprotein Ib b. J Biol Chem 269, 17424-17427.

110. Zhang, L., Eddy, A., Teng, Y. T., Fritzler, M., Kluppel, M., Melet, F., and Bernstein, A. (1995). An immunological renal disease in transgenic mice that overexpress Fli-1, a member of the ets family of transcription factor genes. Mol Cell Biol 15, 6961-6970.

111. Zhang, L., Lemarchandel, V., Romeo, P. H., Ben-David, Y., Greer, P., and Bernstein, A. (1993). The Fli-1 proto-oncogene, involved in erythroleukemia and Ewing's sarcoma, encodes a transcriptional activator with DNA-binding specificities distinct from other Ets family members. Oncogene 8, 1621-1630.

112. Zuker-Franklin, D. (1989). Megakaryocytes and platelets. In Atlas of Blood Cells: Finction and Pathology, D. Zuker-Franklin, Greaves, M. F., Grossi, C. E., and Marmont, A. M., ed. (Philadelphia, Pensylvania, Lea and Feiger), pp. 621-693.

We claim:

1. An isolated nucleic acid molecule of at least 30 nucleotides which hybridizes to SEQ ID NO. 1 or SEQ ID NO.3, or the complement of SEQ ID NO. 1 or SEQ ID NO.3, under stringent hybridization conditions.

2. An isolated nucleic acid molecule as claimed in claim 1 which comprises:
(i) a nucleic acid sequence encoding a polypeptide having substantial sequence identity with the amino acid sequence of SEQ. ID. NO.2 or SEQ. ID. NO 4.;
(ii) nucleic acid sequences complementary to (i);
(iii) a degenerate form of a nucleic acid sequence of (i);
(iv) a nucleic acid sequence comprising at least 18 nucleotides and capable of hybridizing to a nucleic acid sequence in (i), (ii), or (iii);
(v) a nucleic acid sequence encoding a truncation, an analog, an allelic or species variation of a polypeptide comprising the amino acid sequence of SEQ. ID. NO.2, or SEQ. ID. NO 4; or
(vi) a fragment, or allelic or species variation of (i), (ii) or (iii).

3. An isolated nucleic acid molecule as claimed in claim 1 which comprises:
(i) a nucleic acid sequence having substantial sequence identity or sequence similarity with a nucleic acid sequence of SEQ. ID. NO. 1 or 3;
(ii) nucleic acid sequences complementary to (i), preferably complementary to the full nucleic acid sequence of SEQ. ID. NO. 1 or 3;
(iii) nucleic acid sequences differing from any of the nucleic acid sequences of (i) or (ii) in codon sequences due to the degeneracy of the genetic code; or
(iv) a fragment, or allelic or species variation of (i), (ii) or (iii).

4. An isolated nucleic acid molecule as claimed in claim 1 comprising SEQ ID NO. 1 or SEQ ID NO.3.

5. A regulatory sequence of an isolated nucleic acid molecule as claimed in claim 1 fused to a nucleic acid which encodes a heterologous protein.

6. A vector comprising a nucleic acid molecule of claim 1.

7. A host cell comprising a nucleic acid molecule of claim 1.

8. An isolated polypeptide comprising an amino acid sequence of SEQ. ID. NO. 2 or SEQ. ID. NO. 4.

9. A method for preparing a polypeptide as claimed in claim 8 comprising:
(a) transferring a vector as claimed in claim 6 into a host cell;
(b) selecting transformed host cells from untransformed host cells;
(c) culturing a selected transformed host cell under conditions which allow expression of the protein; and
(d) isolating the polypeptide.

10. A polypeptide prepared in accordance with the method of claim 9.

11. An antibody having specificity against an epitope of a polypeptide as claimed in claim 8.

12. A probe comprising a sequence encoding a polypeptide as claimed in claim 8, or a part thereof.

13. A method of diagnosing and monitoring conditions mediated by a polypeptide comprising an amino acid sequence of SEQ. ID. NO. 2 or SEQ. ID. NO. 4 by determining the presence of a nucleic acid molecule as claimed claim 1.

14. A method of diagnosing and monitoring conditions mediated by a polypeptide comprising an amino acid sequence of SEQ. ID. NO. 2 or SEQ. ID. NO. 4 by determining the presence of a polypeptide as claimed in claim 8.

15. A method for identifying a substance which associates with a polypeptide as claimed in claim 8 comprising (a) reacting the polypeptide with at least one substance which potentially can associate with the polypeptide, under conditions which permit the association between the substance and polypeptide, and (b) removing or detecting polypeptide associated with the substance, wherein detection of associated polypeptide and substance indicates the substance associates with the polypeptide.

16. A method for evaluating a compound for its ability to modulate the biological activity of a polypeptide as claimed in claim 8 comprising reacting the polypeptide with a substance which associates with the polypeptide and a test compound under conditions which permit the formation of complexes between the substance and polypeptide, and removing and/or detecting complexes.

17. A method for detecting a nucleic acid molecule encoding a polypeptide as claimed in claim 8 in a biological sample comprising the steps of:
(a) hybridizing a nucleic acid molecule of claim 4 to nucleic acids of the biological sample, thereby forming a hybridization complex; and
(b) detecting the hybridization complex wherein the presence of the hybridization complex correlates with the presence of a nucleic acid molecule encoding the polypeptide in the biological sample.

18. A method for treating a condition mediated by a polypeptide comprising an amino acid sequence of SEQ. ID. NO. 2 or SEQ. ID. NO. 4 comprising administering an effective amount of an antibody as claimed in claim 11.

19. A composition comprising a nucleic acid molecule as claimed in claim 1 and a pharmaceutically acceptable carrier, excipient or diluent.

20. A composition comprising a polypeptide as claimed in claim 8, and a pharmaceutically acceptable carrier, excipient or diluent.

21. A transgenic non-human mammal which does not express or has altered expression of a polypeptide as claimed in claim 8 resulting in a Hzf or Hhl associated pathology.

22. A transgenic animal assay system which provides a model system for testing for an agent that reduces or inhibits an Hzf or Hhl associated pathology comprising
(a) administering the agent to a transgenic non-human animal as claimed in claim 21; and
(b) determining whether said agent reduces or inhibits an Hzf or Hhl associated pathology in the transgenic mouse relative to a transgenic mouse of step (a) which has not been administered the agent.
